(19) United States
(12) Patent Application Publication
(10) Pub. No.: US 2022/0273422 A1
(43) Pub. Date: Sep. 1, 2022

(54) CORNEAL INLAY DESIGN AND METHODS OF CORRECTING VISION

(71) Applicant: RVO 2.0, Inc. D/B/A Optics Medical, Aliso Viejo, CA (US)

(72) Inventors: Nicholas J. Manesis, Aliso Viejo, CA (US); Phihoa Tran-Hata, Aliso Viejo, CA (US); Alan Le, Aliso Viejo, CA (US); Khanh Nguyen, Aliso Viejo, CA (US)

(21) Appl. No.: 17/631,822

(22) PCT Filed: Jul. 30, 2020
(86) PCT No.: PCT/US2020/044266

§ 371 Date: Jan. 31, 2022

Related U.S. Application Data
(60) Provisional application No. 62/881,124, filed on Jul. 31, 2019.

(51) Int. Cl. A61F 2/14; A61L 27/42; A61L 27/26; A61L 27/52

(52) U.S. Cl. CPC A61F 2/1451; A61L 27/427; A61L 27/26; A61L 27/52; A61F 2230/008

(57) ABSTRACT

A corneal inlay device comprising a flat or flat-like base and a dome or droplet top. The corneal inlay can be used to treat, for example without limitation, presbyopia, while reducing or eliminating the risk of a patient developing corneal haze.

### CROSS-REFERENCE TO RELATED APPLICATIONS

This application claims priority to U.S. Provisional Application No. 62/881,124, filed Jul. 31, 2019, entitled “Corneal Inlay Design and Methods to Correcting Vision”. The entire contents of the aforementioned application is incorporated by reference herein in its entirety.

### FIELD OF THE INVENTION

The described invention relates generally to medical devices, and more particularly to corneal inlays.

### BACKGROUND OF THE INVENTION

Parts of the Eye

The eye is an organ which reacts to light and pressure and allows the sense of vision. FIG. 1 is an illustration of a human eye. (Allaboutvision.com/resources/anatomy.htm, Accessed March 2019.) The anatomy of the eye includes a conjunctiva, a iris, a lens, a pupil, a cornea, a sclera, a ciliary body, a vitreous body, a anterior chamber, a choroid, a retina, a macula, a optic nerve, and an optic disc. The conjunctiva is a clear, thin membrane that covers part of the front surface of the eye and the inner surface of the eyelids. The iris is a thin, circular structure made of connective tissue and muscle that surrounds the pupil and regulates the amount of light that strikes the retina. The retina is a light-sensitive membrane on which light rays are focused. It is composed of several layers, including one that contains specialized cells called photoreceptors. Photoreceptor cells take light focused by the cornea and lens (a transparent, biconvex structure) and convert it into chemical and nervous signals which are transported to visual centers in the brain by way of the optic nerve. The sclera is dense connective tissue that that surrounds the cornea and forms the white part of the eye. The ciliary body connects the iris to the choroid and consists of ciliary muscle (which alters the curvature of the lens), a series of radial ciliary processes (from which the lens is suspended by ligaments), and the ciliary ring (which adjoins the choroid). The choroid is the pigmented vascular layer of the eye between the retina and the sclera. The vitreous body is a transparent, colorless, semisolid mass composed of collagen fibrils and hyaluronic acid that fills the posterior cavity of the eye between the lens and the retina. The anterior chamber is an aqueous humor-filled space inside the eye between the iris and the cornea's innermost surface. The macula is an oval-shaped pigmented area near the center of the retina. The optic disc is the raised disk on the retina at the point of entry of the optic nerve, which lacks visual receptors, thus creating a blind spot.

The Cornea

The cornea is a clear and transparent layer anterior on the eye. It is the eye's main refracting surface. FIG. 2 is an illustration showing the cornea, which is avascular and exhibits the following five layers, from the anterior (nearer to the front) to posterior (nearer to the rear) direction, the epithelium, Bowman's layer, stroma, Descemet's membrane, and the endothelium.

The epithelium is a layer of cells that can be thought of as covering the surface of the cornea. Specifically, the cornea is covered externally by a stratified nonkeratinizing epithelium (5-6 layers of cells, about 50 microns in thickness) with three types of cells: superficial cells, wing cells and basal cells (deepest cell layer). Desmosomes form tight junctions in between the superficial cells. The basal cells are the only corneal epithelial cells capable of mitosis; the basement membrane of epithelial cells is 40-60 nm in thickness and is made up of type IV collagen and laminin secreted by basal cells. The epithelial layer is highly sensitive due to numerous nerve endings and has excellent regenerative power. There are differences between epithelium of central and peripheral cornea. In the central cornea, the epithelium has 5-7 layers, the basal cells are columnar; there are no melanocytes or Langerhans cells, and the epithelium is uniform to provide a smooth regular surface. In the peripheral cornea, the epithelium is 7-10 layered, the basal cells are cuboidal, there are melanocytes and Langerhans cells, and there are undulating extensions of the basal layer. (Sridhar, M. S., “Anatomy of cornea and ocular surface,” Indian J. Ophthalmol. (2018) 66(2): 190-194).

Bowman membrane is structureless and acellular. The stroma is the thickest layer of the cornea and gives the cornea much of its strength. Most refractive surgeries involve manipulating stroma cells. Specifically, the substantia propria (stroma) forms 90% of the cornea's thickness and is made up of keratocytes and extracellular matrix. Fibrils of the stroma crisscross at 90° angles; these fibrils are of types I, III, V, and VII collagen.

Descemet's membrane and the endothelium are considered the posterior portion of the cornea. Descemet membrane is structureless, homogeneous, and measures 3-12 microns; it is composed of the anterior banded zone and the posterior nonbanded zone; the Descemet membrane is rich in type IV collagen fibers.

The cornea is covered internally by the corneal endothelium, a single layer, 5 microns thick, of simple cuboidal and hexagonal cells with multiple orthogonal arrays of collagen in between. The endothelium is derived from the neural crest and functions to transport fluid from the anterior chamber to the stroma. Because the cornea is avascular, its nutrients are derived mainly from diffusion from the endothelium layer. (Duong, H-V. Q. “Eye Globe Anatomy,” https://emedicine.medscape.com/article/1923010-Overview, updated Nov. 9, 2017). It is normally avascular due to the high concentration of soluble VEGFR-1, and is surrounded by a transitional margin, the corneal limbus, within which resides nascent endothelium and corneal epithelial stem cells. Id.

Zones of the Cornea

The shape of the cornea is aspheric, meaning that it departs slightly from the spherical form. Typically, the central cornea is about 3D steeper than the periphery. (http://www.aao.org/bcsdsnippetdetail.aspx?id=65c7bff9-4fle-47''7-8585-40318390fc7c, visited 3/12/19)

Clinically, the cornea is divided into zones that surround fixation and blend into one another. The central zone of 1-2 mm closely fits a spherical surface. Adjacent to the central zone is the paracentral zone, a 3-4 mm doughnut with an outer diameter of 7-8 mm that represents an area of progressive flattening from the center. Together, the paracentral and central zones constitute the apical zone. The central and paracentral zones are primarily responsible for the refractive power of the cornea. Adjacent to the paracentral zone is the peripheral zone, with an outer diameter of approximately 11 mm. The peripheral zone is also known as the transitional zone, as it is the area of greatest flattening and asphericity of the normal cornea. Adjoining the peripheral zone is the limbus, with an outer diameter that averages 12 mm. Id.

The optical zone is the portion of the cornea that overlies the entrance pupil of the iris; it is physiologically limited to approximately 5.4 mm because of the Stiles-Crawford effect (the reduction of the brightness when a light beam's entry into the eye is shifted from the center to the edge of the pupil has from the outset been shown to be due to a change in luminous efficiency of radiation when it is incident obliquely on the retina. See G. Westheimer, “Directional sensitivity of the retina: 75 years of Stiles-Crawford effect,” Proc. R. Soc. B. (2008) 275 (1653): 2777-86).Id.

The corneal apex is the point of maximum curvature. The corneal vertex is the point located at the intersection of a subject's line of fixation and the corneal surface.

The cornea must be clear, smooth and healthy for good vision. If it is scarred, swollen, or damaged, light is not focused properly into the eye.

Corneal Wound Healing

The term “wound healing” refers to the process by which the body repairs trauma to any of its tissues, especially those caused by physical means and with interruption of continuity.

A wound-healing response often is described as having three distinct phases-injury, inflammation and repair. Injury often results in the disruption of normal tissue architecture, initiating a healing response. Generally speaking, the body responds to injury with an inflammatory response, which is crucial to maintaining the health and integrity of an organism. The closing phase of wound healing consists of an orchestrated cellular reorganization guided by a fibrin (a fibrous protein that is polymerized to form a “mesh” that forms a clot over a wound site)-rich scaffold formation, wound contraction, closure and re-epithelialization.

The response of the anterior segment of the eye to wound healing closely resembles the response of non-CNS tissues. Friedlander, M. “Fibrosis and diseases of the eye,” J. Clin. Invest. (2007) 117(3): 576-86.

The healing of corneal epithelial wounds involves a number of concerted events, including cell migration, proliferation, adhesion and differentiation, with cell layer stratification.

In brief, corneal epithelial healing largely depends on limbal epithelial stem cells (LESCs) stem cells, which, in many species, including humans, exclusively reside in the corneoscleral junction, and remodeling of the basement membrane. Ljubimov, A V and Saghizadeh, M., “Progress in corneal wound healing,” Prog. Retin. Eye Res. (2015) 49: 17-45. In response to injury, LESCs undergo few cycles of proliferation and give rise to many transit-amplifying cells (TACS), which appear to make up most of the basal epithelium in the limbus and peripheral cornea. Id. The LESCs are thought to migrate into the central cornea, proliferate rapidly afterwards, and eventually terminally differentiate into central corneal epithelial cells. Id. During stromal healing, keratocytes get transformed to motile and contractile myofibroblasts largely due to activation of the transforming growth factor β system. Id. Endothelial cells heal mostly by migration and spreading, with cell proliferation playing a secondary role. Id.

Epithelial Wound Healing

The kinetics of epithelial wound healing includes two distinct phases: an initial latent phase, and a closure phase. The initial latent phase includes cellular and subcellular reorganization to trigger migration of the epithelial cells at the wound edge. (Id., citing Kuwabara T, et al., Sliding of the epithelium in experimental corneal wounds. Invest. Ophthalmol. (1976) 15: 4-14; Crosson, C E et al., Epithelial wound closure in the rabbit cornea. A biphasic process. Invest. Ophthalmol. Vis. Sci. (1986) 27: 464-473). The closure phase includes several continuous processes starting with cell migration, which is independent of cell mitosis. (Id, citing Anderson, S C, et al., Rho and Rho-kinase (ROCK) signaling in adherens and gap junction assembly in corneal epithelium. Invest. Ophthalmol. Vis. Sci. (2002) 43: 978-986), followed by cell proliferation and differentiation, and eventually, by stratification to restore the original multicellular epithelial layer (Id., citing Crosson, C E et al., Epithelial wound closure in the rabbit cornea. A biphasic process. Invest. Ophthalmol. Vis. Sci. (1986) 27: 464-473)).

Wound Healing Factors in Epithelial Wound Healing

When corneal epithelium is injured, nucleotides and neuronal factors are released to the extracellular milieu, generating a Ca(2+) wave from the origin of the wound to neighboring cells. (Id., citing Lee, A, et al., Hypoxia-induced changes in Ca2+ mobilization and protein phosphorylation implicated in impaired wound healing. Am. J. Physiol. Cell. Physiol. (2014) 306: C972-985). The release of ATP induced within one minute after injury results in mobilization of intracellular calcium upon activation of purinergic receptors P2Y or P2X (Id. citing Weinger, I et al Tri-nucleotide receptors play a critical role in epithelial cell wound repair. Purinerg. Signal. (2005) 1: 281-292; Hypoxia-induced changes in Ca2+ mobilization and protein phosphorylation implicated in impaired wound healing. Am. J. Physiol. Cell. Physiol. (2014) 306: C972-985). This activation appears to be one of the earliest events in the healing process. (Id., citing Lee, A. et al, Hypoxia-induced changes in Ca2+ mobilization and protein phosphorylation implicated in impaired wound healing. Am. J. Physiol. Cell. Physiol. (2014) 306: C972-985). Most recent data show that the effects of P2X7 on wound healing may be mediated by a rearrangement of actin cytoskeleton enabling epithelial cells to better migrate (Id.).

Toll-like receptors (TLRs) also contribute to early corneal epithelial wound healing by enhancing cell migration and proliferation in vitro and in vivo (Id., citing Eslani, M et al, The role of toll-like receptor 4 in corneal epithelial wound healing. Invest. Ophthalmol. Vis. Sci. (2014) 55: 6108-6115). TLRs are a family of proteins that play a major role in the innate immune system and modulate inflammation via several pathways, such as nuclear factor κB (NF-κB), MAP kinases, and activator protein (AP)-1. (Id., citing Pearlman, E et al., Toll-like receptors at the ocular surface. Ocul. Surf. (2008) 6: 108-116; Kostarnoy, A V et al., Topical bacterial lipopolysaccharide application affects inflammatory response and promotes wound healing. J. Interferon Cytokine Res. (2013) 33: 514-522). The TLR signaling pathway is activated in response to its ligands, such as pathogen associated molecular patterns (“PAMPs”, for viruses and bacteria) and damage-associated molecular patterns (DAMPs) as a result of tissue injury. This leads to production of proinflammatory cytokines, adhesion molecules and proteolytic enzymes during the inflammatory stage of wound healing. (Id., citing Pearlman, E et al., Toll-like receptors at the ocular surface. Ocul. Surf. (2008) 6: 108-116; Kostarnoy, A V et al., Topical bacterial lipopolysaccharide application affects inflammatory response and promotes wound healing. J. Interferon Cytokine Res. (2013) 33: 514-522) as well as to enhanced cell migration and proliferation.

In the initial or lag phase of wound healing, several parallel signaling pathways, which may cross-talk, are activated to reorganize cellular and subcellular structures initiating cell migration, the first step of the healing process. These initial factors include IL-1 and TNF-α (Id., citing Wilson S E, et al. Stromal-epithelial interactions in the cornea. Prog. Retin. Eye Res. (1999) 18: 293-309), EGF and PDGF (Id., citing Tuominen, I S et al, Human tear fluid PDGF-BB, TNF-α and TGF-β1 vs corneal haze and regeneration of corneal epithelium and subbasal nerve plexus after PRK. Exp. Eye Res. (2001) 72: 631-641), which trigger a series of responses leading to epithelial cell migration through ERK, MAP kinases, and/or NF-κB pathways. Additionally, a number of transcription factors, such c-fos, c-jun, jun-B, and fos-B, which become activated during the lag phase of wound healing before the cells start to migrate (Id., citing Oakdale, Y, Expression of fos family and jun family proto-oncogenes during corneal epithelial wound healing. Curr. Eye Res. (1996) 15: 824-832), can also lead to activation of other parallel pathways in underlying stroma, including IL-1 mediated keratocyte apoptosis via FAS/Fas ligand (Id., citing Wilson S E, et al., Stromal-epithelial interactions in the cornea. Prog. Retin. Eye Res. (1999) 18: 293-309), which leads to consecutive pathways of pro-inflammatory cascades in the first 24 hr following injury (Id., citing Wilson S E, et al., The corneal wound healing response: cytokine mediated interaction of the epithelium, stroma, and inflammatory cells. Prog. Retin. Eye Res. (2001) 20: 625-637). EGFR transactivation has been shown to enhance intracellular signaling in corneal epithelial wound healing in the presence of non-EGF ligands, such as IGF, insulin and HGF by activating ERK and PI3K/Akt pathways (Id., citing Lyu J, Transactivation of EGFR mediates insulin-stimulated ERK1/2 activation and enhanced cell migration in human corneal epithelial cells. Mol. Vis. (2006) 12: 1403-1410; Spix J K, et al., Hepatocyte growth factor induces epithelial cell motility through transactivation of the epidermal growth factor receptor. Exp. Cell Res. (2007) 313: 3319-3325). Hepatocyte growth factor (HGF) and keratinocyte growth factor (KGF), as well as pigment epithelium-derived factor (PEDF) signaling during wound healing converges on p38 and/or ERK1/2 pathways; the former mediates cell migration, whereas the latter induces proliferation (Id., citing Sharma G D, He J, Bazan H E. p38 and ERK1/2 coordinate cellular migration and proliferation in epithelial wound healing: evidence of cross-talk activation between MAP kinase cascades. J. Biol. Chem. (2003) 278: 21989-21997; Ho T C, et al., PEDF promotes self-renewal of limbal stem cell and accelerates corneal epithelial wound healing. Stem Cells. (2013) 31: 1775-1784).

Another initial wound healing factor is the release of matrix metalloproteinases (MMPs), which triggers a series of processes to disengage cell-cell and cell-matrix adhesion. This leads to initiation and facilitation of cell migration via cross-talk with integrins and the production of extracellular matrix (ECM) proteins, such as fibronectin, laminin and tenascin, in the wound area that act as a temporary scaffold for migratory cells (Id., citing Tuft S J, et al., Photorefractive keratectomy: implications of corneal wound healing. Br. J. Ophthalmol. (1993) 77: 243-247). The release of cellular nucleotides (e.g., ATP) upon epithelial injury is also implicated as an initial factor causing rapid activation of purinergic signaling and increase of intracellular CA2+ levels leading to epidermal growth factor receptor (EGFR) transactivation and cell migration, and eventually, epithelial wound healing with corneal nerve involvement (Id., citing Weinger I, et al., Tri-nucleotide receptors play a critical role in epithelial cell wound repair. Purinerg. Signal. (2005) 1: 281-292); Boucher I. Injury and nucleotides induce phosphorylation of epidermal growth factor receptor: MMP and HB-EGF dependent pathway. Exp. Eye Res. (2007) 85: 130-141; Yin J, Xu K, Zhang J, Kumar A, Yu F S. Wound-induced ATP release and EGF receptor activation in epithelial cells. J. Cell Sci. (2007) 120: 815-825; Lee A, et al., Hypoxia-induced changes in Ca2+ mobilization and protein phosphorylation implicated in impaired wound healing. Am. J. Physiol. Cell. Physiol. (2014) 306: C972-985). EGFR and purinergic signaling are also involved in the phosphorylation of paxillin, a focal adhesion-associated phosphotyrosine-containing protein that contains a number of motifs that mediate protein-protein interactions (see Schaller, MD, “Paxillin: a focal adhesion associated adaptor protein,” Oncogene (2001) 20: 6459-72) needed for cell migration (Id., citing Kimura K, et al., Role of JNK-dependent serine phosphorylation of paxillin in migration of corneal epithelial cells during wound closure. Invest. Ophthalmol. Vis. Sci. (2008) 49: 125-132; Mayo C, et al., Regulation by P2X7: epithelial migration and stromal organization in the cornea. Invest. Ophthalmol. Vis. Sci. (2008) 49: 4384-4391).

Cell migration during wound healing also may involve a cross-talk between growth factors and ECM. Insulin-like growth factor 1 (IGF1) was shown to induce cell migration directly through its receptor, as well as through stimulating the expression of corneal basement membrane component laminin-332, which facilitates epithelial cell migration in vitro (Id., citing Lee J G, Kay E P. FGF-2-induced wound healing in corneal endothelial cells requires Cdc42 activation and Rho inactivation through the phosphatidylinositol 3-kinase pathway. Invest. Ophthalmol. Vis. Sci. (2006) 47: 1376-1386). IGF1 receptor can also be engaged in cross-talk with β1 chain-containing integrins important for corneal epithelial cell migration (Id., citing Seomun Y, Joo C K. Lumican induces human corneal epithelial cell migration and integrin expression via ERK 1/2 signaling. Biochem. Biophys. Res. Commun. (2008) 372: 221-225) through their recruitment to lipid rafts (Id., citing Salani B, et al., IGF-I induced rapid recruitment of integrin β1 to lipid rafts is caveolin-1 dependent. Biochem. Biophys. Res. Commun. (2009) 380: 489-492). Overall, significant cross-talk in corneal wound healing has been revealed between several growth factors through transactivation of signaling pathways, and between growth factors and extracellular mediators of this process. This cross-talk underlines the complex nature of epithelial wound healing.

ECM in Epithelial Wound Healing

Corneal epithelium makes its own ECM in the form of a specialized epithelial basement membrane that is positioned between basal epithelial cells and the stroma and apposed to the underlying collagenous Bowman's layer. It provides structural support and regulates, through various receptors, epithelial migration, proliferation, differentiation, adhesion and apoptosis (Id., citing Azar D T, et al., Altered epithelial-basement membrane interactions in diabetic corneas. Arch. Ophthalmol. (1992) 110: 537-40; Kurpakus M A, et al., The role of the basement membrane in differential expression of keratin proteins in epithelial cells. Dev. Biol. (1992) 150: 243-255; Zieske J D, et al., Basement membrane assembly and differentiation of cultured corneal cells: importance of culture environment and endothelial cell interaction. Exp. Cell Res. (1994) 214: 621-633; Ljubimov A V, et al., Extracellular matrix alterations in human corneas with bullous keratopathy. Invest. Ophthalmol. Vis. Sci. (1996) 37: 997-1007; Ljubimov A V, et al., Basement membrane abnormalities in human eyes with diabetic retinopathy. J Histochem Cytochem. (1996) 44: 1469-1479; Suzuki K, et al., Cell-matrix and cell-cell interactions during corneal epithelial wound healing. Prog. Retin. Eye Res. (2003) 22: 113-133). Corneal epithelial basement membrane is composed of specialized networks of type IV collagens, laminins, nidogens and perlecan, as are most basement membranes (Id., citing Nakayasu K, et al., Distribution of types I, II, III, IV and V collagen in normal and keratoconus corneas. Ophthalmic Res. (1986) 18: 1-10; Martin G R, Timpl R. Laminin and other basement membrane components. Annu. Rev. Cell Biol. (1987) 3: 57-85; Ljubimov A V, et al., Human corneal basement membrane heterogeneity: topographical differences in the expression of type IV collagen and laminin isoforms. Lab Invest. (1995) 72: 461-473; Ljubimov A V, et al., Extracellular matrix alterations in human corneas with bullous keratopathy. Invest. Ophthalmol. Vis. Sci. (1996) 37: 997-1007; Tuori A, et al., The immunohistochemical composition of the human corneal basement membrane. Cornea. (1996) 15: 286-294; Kabosova A, et al., Human diabetic corneas preserve wound healing, basement membrane, integrin and MMP-10 differences from normal corneas in organ culture. Exp. Eye Res. (2003) 77: 211-217; Schlötzer-Schrehardt U, et al., Characterization of extracellular matrix components in the limbal epithelial stem cell compartment. Exp. Eye Res. (2007) 85: 845-60) with additional components, such as TSP-1, matrilin-2, matrilin-4, types CV, XCII and XCIII collagen and fibronectin (FN) (Id., citing Kabosova A, et al., Compositional differences between infant and adult human corneal basement membranes. Invest. Ophthalmol. Vis. Sci. (2007) 48: 4989-4999; Schlötzer-Schrehardt U, et al., Characterization of extracellular matrix components in the limbal epithelial stem cell compartment. Exp. Eye Res. (2007) 85: 845-60; Dietrich-Ntoukas T, et al., Comparative analysis of the basement membrane composition of the human limbus epithelium and amniotic membrane epithelium. Cornea. (2012) 31: 564-569).

Immune System Involvement in Epithelial Wound Healing

Immune system cells, such as neutrophils, play a major role in corneal epithelial wound healing, which might be due to their ability to release growth factors that impact the epithelium (Li Z, et al., Lymphocyte function-associated antigen-1-dependent inhibition of corneal wound healing. Am. J. Pathol. (2006) 169: 1590-600; Li Z, et al., Platelet response to corneal abrasion is necessary for acute inflammation and efficient re-epithelialization. Invest. Ophthalmol. Vis. Sci. (2006) 47: 4794-4802).

The major function of corneal epithelium is to protect the eye interior by serving as a physical and chemical barrier against infection by tight junctions and sustaining the integrity and visual clarity of cornea. Id. Wounded, damaged or infected epithelial cells secret the cytokine, IL-1α, which is stored in epithelial cells and released when the cell membrane is damaged by external insults. Id. Secreted IL-1α can cause increased immune infiltration of the cornea leading to neovascularization, which may result in visual loss. Id. However, IL-1RN, an IL-1α antagonist, prevents leucocyte invasion of the cornea and suppresses neovascularization, which may help preserve vision (Id., citing Stapleton W M, et al., Topical interleukin-1 receptor antagonist inhibits inflammatory cell infiltration into the cornea. Exp. Eye Res. (2008) 86: 753-757). In animal models, corneal epithelial wounding prompts an acute inflammatory response in the limbal blood vessels leading to accumulation of leukocytes and neutrophils (Id., citing Li S D, Huang L. Non-viral is superior to viral gene delivery. J. Control Release. (2007) 123: 181-183; Yamagami S, et al., CCR5 chemokine receptor mediates recruitment of MHC class II-positive Langerhans cells in the mouse corneal epithelium. Invest. Ophthalmol. Vis. Sci. (2005) 46: 1201-1207), and migration of dendritic cells, macrophages and lymphocytes (Id., citing Jin Y, et al., The chemokine receptor CCR7 mediates corneal antigen-presenting cell trafficking. Mol. Vis. (2007) 13: 626-634; Li S D, Huang L. Non-viral is superior to viral gene delivery. J. Control Release. (2007) 123: 181-183; Gao N, et al., Dendritic cell-epithelium interplay is a determinant factor for corneal epithelial wound repair. Am. J. Pathol. (2011) 179: 2243-53) into the stroma and the wounded epithelium. Current evidence indicates that the innate inflammatory responses are necessary for corneal epithelial wound healing and nerve recovery (Id., citing Li Z, et al., Lymphocyte function-associated antigen-1-dependent inhibition of corneal wound healing. Am. J. Pathol. (2006) 169: 1590-600; Li S D, Huang L. Non-viral is superior to viral gene delivery. J. Control Release. (2007) 123: 181-183; 2011; Gao N, et al., Dendritic cell-epithelium interplay is a determinant factor for corneal epithelial wound repair. Am. J. Pathol. (2011) 179: 2243-53). Platelets also accumulate in the limbus and migrate to the stroma in response to wounded epithelium, which is necessary for efficient re-epithelialization through cell adhesion molecules such as P-selectin (Id., citing Li Z, et al., Platelet response to corneal abrasion is necessary for acute inflammation and efficient re-epithelialization. Invest. Ophthalmol. Vis. Sci. (2006) 47: 4794-4802; Lam F W, et al., Platelets enhance neutrophil transendothelial migration via P-selectin glycoprotein ligand-1. Am. J Physiol. Heart Circ. Physiol. (2011) 300: H468-H475).

Epithelial Basement Membrane (BM) in Corneal Wound Healing

Several studies have demonstrated the importance of epithelial BM in corneal wound healing. (Torricelli, A A M et al, The Corneal Epithelial Basement Membrane: Structure, Function and Disease, Invest. Ophthalmol. Vis. Sci. (2013) 54: 6390-6400, citing Fujikawa L S, et al., Basement membrane components in healing rabbit corneal epithelial wounds: immunofluorescence and ultrastructural studies. J Cell Biol. (1984) 98: 128-138; Sta Iglesia D D, Stepp M A. Disruption of the basement membrane after corneal debridement. Invest Ophthalmol Vis Sci. (2000) 41: 1045-1053; Chi C, Trinkaus-Randall V. New insights in wound response and repair of epithelium. J Cell Physiol. (2013) 228: 925-929; Pal-Ghosh S, et al., Removal of the basement membrane enhances corneal wound healing. Exp Eye Res. (2011) 93: 927-936). For example, Pal-Ghosh and coworkers demonstrated that removal of the epithelial BM enhances many wound healing processes in the cornea, including keratocyte apoptosis and nerve death. (Id., citing Pal-Ghosh S, et al, Removal of the basement membrane enhances corneal wound healing. Exp Eye Res. (2011) 93: 927-936). Corneal surgery, injury, or infection frequently triggers the appearance of stromal myofibroblasts associated with persistent corneal opacity (haze). (Torricelli A A, et al., Transmission electron microscopy analysis of epithelial basement membrane repair in rabbit corneas with haze. Invest Ophthalmol Vis Sci. (2013) 54: 4026-4033) The opacity develops as a result of diminished transparency of the cells themselves and the production of disordered extracellular matrix components by stromal cells. (Id., citing Jester J V, et al., Transforming growth factor (beta)-mediated corneal myofibroblast differentiation requires actin and fibronectin assembly. Invest Ophthalmol Vis Sci. (1999) 40: 1959-1967; Masur S K, et al., Myofibroblasts differentiate from fibroblasts when plated at low density. Proc Natl Acad Sci USA. (1996) 93: 4219-4223; Wilson S E, et al., Stromal-epithelial interactions in the cornea. Prog Retin Eye Res. (1999) 18: 293-309). Singh et al. reported that the normally functioning epithelial BM critically modulates myofibroblast development through its barrier function preventing penetration of epithelial TGF-β1 and platelet-derived growth factor (PDGF) into the stroma at sufficient levels to drive myofibroblast development and maintain viability once mature myofibroblasts are generated. (Id., citing Singh V, et al., Stromal fibroblast-bone marrow-derived cell interactions: implications for myofibroblast development in the cornea. Exp Eye Res. (2012) 98: 1-8). This hypothesis holds that stromal surface irregularity after photorefractive keratectomy (PRK) or other cornea injury leads to structural and functional defects in the regenerated epithelial BM, which increases and prolongs penetration of epithelial TGF-β1 and PDGF into the anterior corneal stroma to promote myofibroblast development from either keratocyte-derived or bone marrow-derived precursor cells. (Id., citing Singh V, et al. Effect of TGFbeta and PDGF-B blockade on corneal myofibroblast development in mice. Exp Eye Res. (2011) 93: 810-817, and Singh, V, Wilson, S E, unpublished data, 2013).

The working hypothesis was that prominent mature myofibroblast generation and resulting disorganized extracellular matrix excretion in the anterior stroma of corneas with significant injury interfere with keratocyte contribution of critical components to the BM (collagen type VII, for example) that results in defective epithelial BM regeneration. Id. Only when the epithelial BM is finally regenerated, which may take years in some corneas with haze, and epithelium-derived TGF-β1 levels fall, do myofibroblasts undergo apoptosis and keratocytes reabsorb disorganized extracellular matrix and thereby restore transparency. (Id., citing Singh V, et al., Stromal fibroblast-bone marrow-derived cell interactions: implications for myofibroblast development in the cornea. Exp Eye Res. (2012) 98: 1-8; Fini M E, Stramer B M. How the cornea heals: cornea-specific repair mechanisms affecting surgical outcomes. Cornea. (2005) 24: S2-S11; Stramer B M, et al., Molecular mechanisms controlling the fibrotic repair phenotype in cornea: implications for surgical outcomes. Invest Ophthalmol Vis Sci. (2003) 44: 4237-4246. Thus, the epithelial BM likely functions as a corneal regulatory structure that limits the fibrotic response in the stroma by modulating the availability of epithelium-derived TGF-β1, PDGF, and perhaps other growth factors and extracellular matrix components, to stromal cells, including myofibroblast precursors. Id. It may also regulate levels of stromal cell-produced epithelial modulators of motility, proliferation, and differentiation like keratinocyte growth factor (KGF) that transition through the BM in the opposite direction. (Id. Citing Wilson S E, et al., Hepatocyte growth factor, keratinocyte growth factor, their receptors, fibroblast growth factor receptor-2, and the cells of the cornea. Invest Ophthalmol Vis Sci. (1993) 34: 2544-2561; Wilson S E, et al. Effect of epidermal growth factor, hepatocyte growth factor, and keratinocyte growth factor, on proliferation, motility and differentiation of human corneal epithelial cells. Exp Eye Res. (1994) 59: 665-678. Thus, corneal epithelial BM may modulate epithelial-to-stroma and stroma-to-epithelial interactions by regulating cytokines and growth factor movement from one cell layer to the other. Id.

Latvala et al. observed that the distribution of α6 and β4 integrins adjacent to the BM changes during epithelial wound healing after epithelial abrasion in the rabbit cornea. (Id., citing Latvala T, et al, Distribution of alpha 6 and beta 4 integrins following epithelial abrasion in the rabbit cornea. Acta Ophthalmol Scand. (1996) 74: 21-25). Stepp et al. have demonstrated that the re-epithelialization of small wounds is accompanied by increased α6β4 integrin. (Id., citing Stepp M A, et al., Changes in beta 4 integrin expression and localization in vivo in response to corneal epithelial injury. Invest Ophthalmol Vis Sci. (1996) 37: 1593-1601). Epithelial cell migration is also affected by the distribution of laminin and collagen IV during corneal wound healing and BM regeneration. (Id., citing Fujikawa L S, et al., Basement membrane components in healing rabbit corneal epithelial wounds: immunofluorescence and ultrastructural studies. J Cell Biol. (1984) 98: 128-138.) Thus, α3(IV) and α4(IV) collagen chains may be important for the healthy corneal epithelium. Id. Upon injury, the BM is remodeled to include α1(IV) and α2(IV) collagen, recapitulating corneal epithelial expression during development. Id.

Corneal Stromal Wound Healing

Stromal remodeling occurs upon direct damage to the stroma and its cells (as exemplified by photorefractive keratectomy (PRK) and LASIK (used for myopia correction)) and upon death of stromal cells (keratocytes) caused by damage to or removal of corneal epithelium by various physical or chemical factors (Id., citing Nakayasu K. Stromal changes following removal of epithelium in rat cornea. Jpn. J. Ophthalmol. (1988) 32: 113-125; Szerenyi K D, et al., Keratocyte loss and repopulation of anterior corneal stroma after de-epithelialization. Arch. Ophthalmol. (1994) 112: 973-976; Wilson S E, et al., Epithelial injury induces keratocyte apoptosis: hypothesized role for the interleukin-1 system in the modulation of corneal tissue organization and wound healing. Exp. Eye Res. (1996) 62:325-327; Wilson S E, et al, The corneal wound healing response: cytokine mediated interaction of the epithelium, stroma, and inflammatory cells. Prog. Retin. Eye Res. (2001) 20: 625-637; Wilson S E, et al., Apoptosis in the initiation, modulation and termination of the corneal wound healing response. Exp. Eye Res. (2007) 85: 305-311). Such damage triggers a release of inflammatory cytokines from epithelial cells and/or tears (Id., citing Maycock N J, Marshall J. Genomics of corneal wound healing: a review of the literature. Acta Ophthalmol. (2014) 92: e170-84), mainly IL-1 (α and β), that cause rapid apoptosis through Fas/Fas ligand system and later, necrosis of mainly anterior keratocytes. These cells die preferentially directly beneath the epithelial wounds, rather than also beyond their edges. The following stromal remodeling with replenishment of these cells from the areas adjacent to the depleted one (Id., citing Zieske J D, et al., Activation of epidermal growth factor receptor during corneal epithelial migration. Invest. Ophthalmol. Vis. Sci. (2000) 41: 1346-1355), also constitutes a wound healing process and may result in fibrotic changes, especially if the epithelial basement membrane was initially damaged (Id., citing Stramer B M, et al., Molecular mechanisms controlling the fibrotic repair phenotype in cornea: implications for surgical outcomes. Invest. Ophthalmol. Vis. Sci. (2003) 44:4237-4246; Fini M E, Stramer B M. How the cornea heals: cornea-specific repair mechanisms affecting surgical outcomes. Cornea. (2005) 24(Suppl 1): S2-S11; West-Mays J A, Dwivedi D J. The keratocyte: corneal stromal cell with variable repair phenotypes. Int. J. Biochem. Cell Biol. (2006) 38: 1625-1631. This is a classical example of how stromal-epithelial interactions influence wound healing process by paracrine mediators. Id.

At the early stage of wound repair, quiescent keratocytes at the wound edges change their properties to become activated to fibroblasts. These cells enter the cell cycle and acquire migratory properties necessary to repopulate and close the wound (Id., citing West-Mays J A, Dwivedi D J. The keratocyte: corneal stromal cell with variable repair phenotypes. Int. J. Biochem. Cell Biol. (2006) 38: 1625-1631). In culture, this transformation is mediated by some (fibroblast growth factor 2 (FGF-2) and PDGF-AB, TGF-β) growth factors, whereas others (IL-1, IGF-1) only confer mitogenic activity (Id., citing Jester J V, Ho-Chang J. Modulation of cultured corneal keratocyte phenotype by growth factors/cytokines control in vitro contractility and extracellular matrix contraction. Exp. Eye Res. (2003) 77: 581-592; Chen J, et al., Rho-mediated regulation of TGF-β1- and FGF-2-induced activation of corneal stromal keratocytes. Invest. Ophthalmol. Vis. Sci. (2009) 50: 3662-3670). These cells remodel their actin cytoskeleton to acquire stress fibers and change their morphology from stellate to an elongated one (Id., citing Jester J V, Ho-Chang J. Modulation of cultured corneal keratocyte phenotype by growth factors/cytokines control in vitro contractility and extracellular matrix contraction. Exp. Eye Res. (2003) 77: 581-592). Fibroblasts downregulate the expression of differentiated keratocyte proteins, such as corneal crystallins (transketolase and aldehyde dehydrogenase 1A1), and keratan sulfate proteoglycans, and start producing proteinases (mostly MMPs), needed to remodel the wound ECM (Id., citing Fini M E. Keratocyte and fibroblast phenotypes in the repairing cornea. Prog. Retin. Eye Res. (1999) 18: 529-551; Jester J V, et al., Corneal stromal wound healing in refractive surgery: the role of myofibroblasts. Prog. Retin. Eye Res. (1999) 18: 311-356; Carlson E C, et al., Altered KSPG expression by keratocytes following corneal injury. Mol. Vis. (2003) 9: 615-623; West-Mays J A, Dwivedi D J. The keratocyte: corneal stromal cell with variable repair phenotypes. Int. J. Biochem. Cell Biol. (2006) 38: 1625-1631).

After they reach the wound bed, fibroblasts start expressing α-smooth muscle actin (α-SMA) and desmin, upregulate the expression of vimentin (Id., citing Chaurasia S S, et al., “Dynamics of the expression of intermediate filaments vimentin and desmin during myofibroblast differentiation after corneal injury” Exp. Eye Res. (2009) 89: 590-59), and become highly motile and contractile myofibroblasts needed to remodel wound ECM and contract the wound. They also deposit provisional ECM rich in fibronectin and some other proteins including tenascin-C and type III collagen (Id., citing Tervo K, et al., Expression of tenascin and cellular fibronectin in the rabbit cornea after anterior keratectomy. Immunohistochemical study of wound healing dynamics. Invest. Ophthalmol. Vis. Sci. (1991) 32: 2912-2918; Fini M E. Keratocyte and fibroblast phenotypes in the repairing cornea. Prog. Retin. Eye Res. (1999) 18: 529-551). Myofibroblasts generate contractile forces to close the wound gap, and the expression of α-SMA directly correlates with corneal wound contraction (Id., citing Jester J V, et al., Expression of alpha-smooth muscle (α-SM) actin during corneal stromal wound healing. Invest. Ophthalmol. Vis. Sci. (1995) 36: 809-819). When the wound does not really contract as in the case of PRK or phototherapeutic keratectomy (PTK), the appearance of myofibroblasts is delayed, and they start accumulating as late as four weeks after irregular PTK (Id., citing Barbosa F L, et al., Corneal myofibroblast generation from bone marrow-derived cells. Exp. Eye Res. (2010) 91:92-96).

It is generally accepted that myofibroblast transformation is triggered by transforming growth factor beta (TGF-β) in vivo, which has been confirmed by numerous studies in vitro (Id., citing Jester J V, et al., Corneal stromal wound healing in refractive surgery: the role of myofibroblasts. Prog. Retin. Eye Res. (1999) 18: 311-356). More recent work has also implicated a potent mitogen, PDGF (both AA and BB) in this process, with the combination of TGF-β and PDGF being more potent than either factor alone (Id., citing Kaur H, et al., Corneal stroma PDGF blockade and myofibroblast development. Exp Eye Res. (2009) 88: 960-965; Singh V, et al., Transforming growth factor β and platelet-derived growth factor modulation of myofibroblast development from corneal fibroblasts in vitro. Exp. Eye Res. (2014) 120: 152-160). Only TGF-β1 and TGF-β2 are active in this process, since TGF-β3 does not transform fibroblasts to myofibroblasts (Id., citing Karamichos D, et al., Reversal of fibrosis by TGF-β3 in a 3D in vitro model. Exp. Eye Res. (2014) 124: 31-36). Upon completion of wound healing, myofibroblasts apparently cease to express α-SMA. Their fate in vivo is not completely clear, Although myofibroblast appearance is widely considered as a necessary part of stromal wound healing, the numbers of myofibroblasts in the corneal stroma after PRK differ widely among various mouse strains (Id., citing Singh V, et al., Mouse strain variation in SMA(+) myofibroblast development after corneal injury. Exp. Eye Res. (2013) 115: 27-30). Some data indicate that repopulation of keratocytes after epithelial debridement of mouse corneas occurs without the appearance of myofibroblasts (Id., citing Matsuba M, et al., Localization of thrombospondin-1 and myofibroblasts during corneal wound repair. Exp. Eye Res. (2011) 93: 534-540), possibly through stimulation of keratocyte migration by aquaporin-1 water channel (Id., citing Ruiz-Ederra J, Verkman A S. Aquaporin-1-facilitated keratocyte migration in cell culture and in vivo corneal wound healing models. Exp. Eye Res. (2009) 89: 159-165).

Immune Cells in Stromal Healing

Corneal injury in animal models entails an inflammatory response by immune system cells, including monocytes/macrophages, T cells, polymorphonuclear (PMN) leukocytes and natural killer (NK) cells (Id., citing Gan L, et al., Effect of leukocytes on corneal cellular proliferation and wound healing. Invest. Ophthalmol. Vis. Sci. (1999) 40: 575-581; Wilson S E, et al., The corneal wound healing response: cytokine mediated interaction of the epithelium, stroma, and inflammatory cells. Prog. Retin. Eye Res. (2001) 20: 625-637; Wilson S E, et al., RANK, RANKL, OPG, and M-CSF expression in stromal cells during corneal wound healing. Invest. Ophthalmol. Vis. Sci. (2004) 45: 2201-2211; Liu Q, et al., NK Cells Modulate the Inflammatory Response to Corneal Epithelial Abrasion and Thereby Support Wound Healing. J. Pathol. (2012) 181: 452-462; Li S, et al., Macrophage depletion impairs corneal wound healing after autologous transplantation in mice. PLoS One. (2013) 8: e61799). These infiltrating cells are usually defined by staining for CD11b, although in some studies a better characterization of these cells is provided (Id., citing Wilson S E, et al., The corneal wound healing response: cytokine mediated interaction of the epithelium, stroma, and inflammatory cells. Prog. Retin. Eye Res. (2001) 20: 625-637; Liu Q, et al., NK Cells Modulate the Inflammatory Response to Corneal Epithelial Abrasion and Thereby Support Wound Healing. J. Pathol. (2012) 181: 452-462; Li S, et al., Macrophage depletion impairs corneal wound healing after autologous transplantation in mice. PLoS One. (2013) 8: e61799). Immune cells may come to the injured cornea from the limbal area or are mobilized from circulation (Id., citing Wilson S E, et al., The corneal wound healing response: cytokine mediated interaction of the epithelium, stroma, and inflammatory cells. Prog. Retin. Eye Res. (2001) 20: 625-637). A major attracting signal for such cells may be monocyte chemotactic protein-1 (MCP-1), a cytokine, which can be secreted by activated fibroblasts and triggered by IL-1 or TNF-α (Id., citing Wilson S E, et al., The corneal wound healing response: cytokine mediated interaction of the epithelium, stroma, and inflammatory cells. Prog. Retin. Eye Res. (2001) 20: 625-637). Another factor required for neutrophil influx following injury was identified as a stromal proteoglycan lumican (Id., citing Hayashi Y, et al., Lumican is required for neutrophil extravasation following corneal injury and wound healing. J. Cell Sci. (2010) 123: 2987-2995). It is still unclear what are the magnitude, infiltrating cell repertoire and origin, as well as kinetics of the immune response to corneal injury in humans.

Functions of immune cells infiltrating injured corneas may be diverse. They may scavenge remnants of apoptotic keratocytes and protect the cornea from possible infection (Id., citing Wilson S E, et al., The corneal wound healing response: cytokine mediated interaction of the epithelium, stroma, and inflammatory cells. Prog. Retin. Eye Res. (2001) 20: 625-637). Some of these cells may become myofibroblasts (Id., citing Barbosa F L, et al., Corneal myofibroblast generation from bone marrow-derived cells. Exp. Eye Res. (2010) 91: 92-96) and thus participate in wound contraction. Direct involvement of immune cells in the wound healing has been also suggested from recent studies. Blocking PMN entry into cornea by fucoidin (inhibitor of leucocyte adhesion to vascular endothelium) delayed wound healing after PRK in rabbits (Id., citing Gan L, et al., Effect of leukocytes on corneal cellular proliferation and wound healing. Invest. Ophthalmol. Vis. Sci. (1999) 40: 575-581). Functional blocking of NK cells after epithelial abrasion and keratocyte loss inhibited healing and nerve regeneration (Id., citing Liu Q, et al., NK Cells Modulate the Inflammatory Response to Corneal Epithelial Abrasion and Thereby Support Wound Healing. J. Pathol. (2012) 181: 452-462). Macrophage depletion impaired wound healing after autologous corneal transplantation, with a decrease in wound myofibroblasts (Id., citing Li S, et al., Macrophage depletion impairs corneal wound healing after autologous transplantation in mice. PLoS One. (2013) 8: e61799). These studies emphasize the importance of local and systemic immunity in corneal wound healing, both epithelial and stromal.

Remodeling of Stromal ECM During Wound Healing

As described above, stromal wound healing is accompanied by several events that may be responsible for ECM changes in this location: death of keratocytes, secretion of proinflammatory and profibrotic cytokines including IL-1, TNF-α, and MCP-1, transient appearance of cells that do not normally form the stroma (PMNs, macrophages, myofibroblasts), and production of ECM-degrading enzymes by activated cells. All these factors contribute to ECM remodeling including its degradation, expression of ectopic components (provisional matrix formation by new cell types), and reassembly of the new ECM to form a more or less normal structure (Id., citing Zieske J D, et al., Kinetics of keratocyte proliferation in response to epithelial debridement. Exp. Eye Res. (2001) 72: 33-39; Torricelli A A, Wilson S E. Cellular and extracellular matrix modulation of corneal stromal opacity. Exp. Eye Res. (2014) 129: 151-160). As a result, new ECM formed during wound healing often accumulates aberrant proteins, both in composition and structure. Over time, these proteins may form local scars persisting for a long time (Id., citing Ishizaki M, et al. Expression of collagen I, smooth muscle alpha-actin, and vimentin during the healing of alkali-burned and lacerated corneas. Invest. Ophthalmol. Vis. Sci. (1993) 34: 3320-3328; Ishizaki M, et al., Stromal fibroblasts are associated with collagen IV in scar tissues of alkali-burned and lacerated corneas. Curr. Eye Res. (1997) 16: 339-348; Maguen E, et al., Alterations of corneal extracellular matrix after multiple refractive procedures: a clinical and immunohistochemical study. Cornea. (1997) 16: 675-682; Ljubimov A V, et al., Extracellular matrix changes in human corneas after radial keratotomy. Exp. Eye Res. (1998) 67: 265-272; Maguen E, et al., Extracellular matrix and matrix metalloproteinase changes in human corneas after complicated laser-assisted in situ keratomileusis (LASIK) Cornea. (2002) 21: 95-100; Maguen E, et al., Immunohistochemical evaluation of two corneal buttons with post-LASIK keratectasia. Cornea. (2007) 26: 983-991; Kato T, et al., Expression of type XVIII collagen during healing of corneal incisions and keratectomy wounds. Invest. Ophthalmol. Vis. Sci. (2003) 44: 78-85; Kamma-Lorger C S, et al., Collagen ultrastructural changes during stromal wound healing in organ cultured bovine corneas. Exp. Eye Res. (2009) 88: 953-959; Torricelli A A, Wilson S E. Cellular and extracellular matrix modulation of corneal stromal opacity. Exp. Eye Res. (2014) 129: 151-160). Because of slow turnover of ECM proteins, the unusual scar components may still be present around the healed wounds for years, especially in human corneas (Id., citing Latvala T, et al., Expression of cellular fibronectin and tenascin in the rabbit cornea after excimer laser photorefractive keratectomy: a 12 month study. Br. J. Ophthalmol. (1995) 79: 65-69; Maguen E, et al., Extracellular matrix and matrix metalloproteinase changes in human corneas after complicated laser-assisted in situ keratomileusis (LASIK) Cornea. (2002) 21: 95-10; Maguen E, et al., Immunohistochemical evaluation of two corneal buttons with post-LASIK keratectasia. Cornea. (2007) 26: 983-991; Maguen E, et al., Alterations of extracellular matrix components and proteinases in human corneal buttons with INTACS for post-laser in situ keratomileusis keratectasia and keratoconus. Cornea. (2008) 27: 565-573). These components, which are normally scarce in or absent from adult corneal stroma, include type III, VIII, XIV, and XVIII collagen, limbal isoforms of type IV collagen, embryonic fibronectin isoforms, thrombospondin-1 (TSP-1), tenascin-C, fibrillin-1, and hevin (an ECM-associated secreted glycoprotein belongin to the secreted protein acidic and rich in cysteine (SPARC) family of matricellular proteins (Id., citing Saika S, et al., Epithelial basement membrane in alkali-burned corneas in rats. Immunohistochemical study. Cornea. (1993) 12: 383-390; Melles G R, et al., Immunohistochemical analysis of unsutured and sutured corneal wound healing. Curr. Eye Res. (1995) 14: 809-817; Nickeleit V, et al., Healing corneas express embryonic fibronectin isoforms in the epithelium, subepithelial stroma, and endothelium. Am. J. Pathol. (1996) 149: 549-558; Ishizaki M, et al., Stromal fibroblasts are associated with collagen IV in scar tissues of alkali-burned and laceraed corneas. Curr. Eye Res. (1997) 16: 339-348; Maguen E, et al., Alterations of corneal extracellular matrix after multiple refractive procedures: a clinical and immunohistochemical study. Cornea. (1997) 16: 675-682; Ljubimov A V, et al., Extracellular matrix changes in human corneas after radial keratotomy. Exp. Eye Res. (1998) 67: 265-272; Zieske J D, et al., Kinetics of keratocyte proliferation in response to epithelial debridement. Exp. Eye Res. (2001)72:33-39; Maguen E, et al., Extracellular matrix and matrix metalloproteinase changes in human corneas after complicated laser-assisted in situ keratomileusis (LASIK) Cornea. (2002) 21: 95-10; Maguen E, et al., Immunohistochemical evaluation of two corneal buttons with post-LASIK keratectasia. Cornea. (2007) 26: 983-991; Maguen E, et al., Alterations of extracellular matrix components and proteinases in human corneal buttons with INTACS for post-laser in situ keratomileusis keratectasia and keratoconus. Cornea. (2008) 27: 565-573; Kato T, et al., Expression of type XVIII collagen during healing of corneal incisions and keratectomy wounds. Invest. Ophthalmol. Vis. Sci. (2003) 44: 78-85; Javier J A, et al., Basement membrane and collagen deposition after laser subepithelial keratomileusis and photorefractive keratectomy in the leghorn chick eye. Arch. Ophthalmol. (2006) 124: 703-709; Matsuba M, et al., Localization of thrombospondin-1 and myofibroblasts during corneal wound repair. Exp. Eye Res. (2011) 93:534-540; Chaurasia S S, et al., Hevin plays a pivotal role in corneal wound healing. PLoS One. (2013) 8: e81544; Saika S, et al., Wakayama symposium: modulation of wound healing response in the corneal stroma by osteopontin and tenascin-C. Ocul. Surf. (2013) 11:12-15); Sullivan, M N, and Sage, E H, Hevin/SCI, a matricellular glycoprotein and potential tumor suppressor of the SPARC/BM-40/Osteonectin family. Intl. J. Biochem. Cell Biol. (2004) 38 (6): 991-96).

Signaling Pathways Associated with Stromal Wound Healing

Keratocyte activation to fibroblasts is mediated by FGF-2, TGF-β, and PDGF, and their proliferation, by EGF, HGF, KGF, PDGF, IL-1 and IGF-I (Id., Citing Stern M E, et al., Effect of platelet-derived growth factor on rabbit corneal wound healing. Wound Repair Regen. (1995) 3: 59-65; Baldwin H C, Marshall J. Growth factors in corneal wound healing following refractive surgery: A review. Acta. Ophthalmol. Scand. (2002) 80: 238-247; Jester J V, Ho-Chang J. Modulation of cultured corneal keratocyte phenotype by growth factors/cytokines control in vitro contractility and extracellular matrix contraction. Exp. Eye Res. (2003) 77: 581-592; Carrington L M, Boulton M. Hepatocyte growth factor and keratinocyte growth factor regulation of epithelial and stromal corneal wound healing. J. Cataract Refract. Surg. (2005) 31: 412-423; Chen J, et al., Rho-mediated regulation of TGF-β1- and FGF-2-induced activation of corneal stromal keratocytes. Invest. Ophthalmol. Vis. Sci. (2009) 50: 3662-3670). Although TGF-β is key to fibroblast to myofibroblast transformation, it actually inhibits keratocyte proliferation and migration (Id., citing Baldwin H C, Marshall J. Growth factors in corneal wound healing following refractive surgery: A review. Acta. Ophthalmol. Scand. (2002) 80: 238-247). Stromal cellular infiltration upon injury was found to be stimulated by such cytokines as MCP-1 and platelet-activating factor (PAF) (Id., citing Wilson S E, et al., The corneal wound healing response: cytokine mediated interaction of the epithelium, stroma, and inflammatory cells. Prog. Retin. Eye Res. (2001) 20: 625-637; Kakazu A, et al., Lipoxin A inhibits platelet-activating factor inflammatory response and stimulates corneal wound healing of injuries that compromise the stroma. Exp. Eye Res. (2012) 103:9-16). As noted above, TGF-β isoforms 1 and 2 (Id., citing Torricelli A A, Wilson S E. Cellular and extracellular matrix modulation of corneal stromal opacity. Exp. Eye Res. (2014) 129: 151-160), as well as bone morphogenetic protein 1 (BMP-1), which is capable of inducing formation of cartilage in vivo (Id., citing Malecaze F, et al., Upregulation of bone morphogenetic protein-1/mammalian tolloid and procollagen C-proteinase enhancer-1 in corneal scarring. Invest. Ophthalmol. Vis. Sci. (2014) 55: 6712-6721) may be responsible for myofibroblast emergence, wound contraction and fibrotic scar formation. TGF-β also promotes deposition of excessive ECM in the wound bed that may result in scar formation directly, as well as by stimulating production of other factors including connective tissue growth factor (CTGF) and IGF-I (Id., citing Izumi K, et al., Involvement of insulin-like growth factor-I and insulin-like growth factor binding protein-3 in corneal fibroblasts during corneal wound healing. Invest. Ophthalmol. Vis. Sci. (2006) 47:591-598; Shi L, et al., Activation of JNK signaling mediates connective tissue growth factor expression and scar formation in corneal wound healing. PLoS One. (2012) 7: e32128; Karamichos D, et al., Reversal of fibrosis by TGF-β3 in a 3D in vitro model. Exp. Eye Res. (2014) 124: 31-36; Torricelli A A, Wilson S E. Cellular and extracellular matrix modulation of corneal stromal opacity. Exp. Eye Res. (2014) 129: 151-160). Therefore, attenuation of TGF-β expression and signaling may provide means to counteract fibrotic changes. For instance, topical rosiglitazone, a ligand of peroxisome proliferator activated receptor γ (PPAR-γ) reduced α-SMA expression and scarring in cat corneas upon excimer laser ablation of anterior stroma without compromising wound healing. In corneal fibroblast cultures, it also counteracted TGF-β induced myofibroblast transformation (Id., citing Huxlin K R, et al., Topical rosiglitazone is an effective anti-scarring agent in the cornea. PLoS One. (2013) 8: e70785). Similar effects were seen with neutralizing antibody to TGF-β (Id., citing Møller-Pedersen T, et al., Neutralizing antibody to TGFβ modulates stromal fibrosis but not regression of photoablative effect following PRK. Curr. Eye Res. (1998) 17: 736-747). Inhibition of JNK signaling suppressed TGF-β induced CTGF expression and scarring in penetrating corneal wounds (Id., citing Shi L, et al., Activation of JNK signaling mediates connective tissue growth factor expression and scar formation in corneal wound healing. PLoS One. (2012) 7: e32128). Inhibitors of mechanistic target of rapamycin (mTOR) and p38 MAP kinase signaling were able to markedly reduce the expression of α-SMA and collagenase in corneal cells and injured corneas (Id., citing Jung J C, et al., Constitutive collagenase-1 synthesis through MAPK pathways is mediated, in part, by endogenous IL-1α during fibrotic repair in corneal stroma. J. Cell Biochem. (2007) 102: 453-462; Huh M I, et al., Distribution of TGF-β isoforms and signaling intermediates in corneal fibrotic wound repair. J. Cell Biochem. (2009) 108: 476-488; Milani B Y, et al., Rapamycin inhibits the production of myofibroblasts and reduces corneal scarring after photorefractive keratectomy. Invest. Ophthalmol. Vis. Sci. (2013) 54: 7424-7430). In alkali-burned corneas, blocking of VEGF by neutralizing antibody bevacizumab also inhibited TGF-β expression and improved corneal transparency (Id., citing Lee S H, et al., Bevacizumab accelerates corneal wound healing by inhibiting TGF-β2 expression in alkali-burned mouse cornea. BMB Rep. (2009) 42: 800-805).

Corneal Endothelial Wound Healing

Due to the relative inaccessibility of corneal endothelial layer, there are fewer studies of endothelial healing. This process mostly occurs as a consequence of various burns (Id., citing Zhao B, et al., An investigation into corneal alkali burns using an organ culture model. Cornea. (2009) 28: 541-546.) and surgeries meant to replace dysfunctional endothelial cells (Descemet's stripping endothelial keratoplasty, DSEK) or endothelial cells with Descemet's membrane (Descemet's membrane endothelial keratoplasty, DMEK) (Id., citing Melles G R, et al., Descemet membrane endothelial keratoplasty (DMEK) Cornea. (2006) 25: 987-990; Price M O, Price F W. Descemet's stripping endothelial keratoplasty. Curr. Opin. Ophthalmol. (2007) 18: 290-294. 2007; Caldwell M C, et al., The histology of graft adhesion in Descemet stripping with endothelial keratoplasty. Am. J. Ophthalmol. (2009) 148: 277-281; Dirisamer M, et al., Patterns of corneal endothelialization and corneal clearance after Descemet membrane endothelial keratoplasty for Fuchs endothelial dystrophy. Am. J. Ophthalmol. (2011) 152: 543-555). The wound healing process of corneal endothelium has certain peculiarities. In many tissues, this process entails cell proliferation as a major mechanism of reducing and remodeling the wound bed. However, corneal endothelial cells, especially human, have very low proliferation rates (Id., citing Mimura T, et al., Corneal endothelial regeneration and tissue engineering. Prog. Retin. Eye Res. (2013) 35: 1-17). It is generally considered that corneal endothelium closes the wound gap mainly by migration and increased cell spreading. These two processes are pharmacologically separable and, depending on the wound nature, their relative contribution may vary (Id., citing Joyce N C, et al., In vitro pharmacologic separation of corneal endothelial migration and spreading responses. Invest. Ophthalmol. Vis. Sci. (1990) 31: 1816-1826.; Ichijima H, et al., Actin filament organization during endothelial wound healing in the rabbit cornea: comparison between transcorneal freeze and mechanical scrape injuries. Invest. Ophthalmol. Vis. Sci. (1993) 34: 2803-28012; Gordon S R. Cytological and immunocytochemical approaches to the study of corneal endothelial wound repair. Prog. Histochem. Cytochem. (1994) 28: 1-64; Mimura T, et al., Corneal endothelial regeneration and tissue engineering. Prog. Retin. Eye Res. (2013) 35: 1-17). Some data suggest that during healing, cell division remains very low (Id., citing Lee J G, Kay E P. FGF-2-induced wound healing in corneal endothelial cells requires Cdc42 activation and Rho inactivation through the phosphatidylinositol 3-kinase pathway. Invest. Ophthalmol. Vis. Sci. (2006) 47:1 376-1386), although this view is challenged by the fact that healing corneal endothelial cells mostly divide amitotically, with formation of binuclear cells (Id., citing Landshman N, et al., Cell division in the healing of the corneal endothelium of cats. Arch. Ophthalmol. (1989) 107: 1804-1808).

Endothelial wound healing is associated with a transient acquisition of fibroblastic morphology and actin stress fibers by migrating cells, which is consistent with endothelial-mesenchymal transformation (EnMT) (Id., citing Lee H T, et al., FGF-2 induced by interleukin-1 beta through the action of phosphatidylinositol 3-kinase mediates endothelial mesenchymal transformation in corneal endothelial cells. J. Biol. Chem. (2004) 279: 32325-32332; Miyamoto T, et al., Endothelial mesenchymal transition: a therapeutic target in retrocorneal membrane. Cornea. (2010) 29(Suppl 1): S52-56). In a model of freeze injury, EnMT to myofibroblasts occurs at the migrating front, where cells lose tight junction protein ZO-1 and start expressing α-SMA (Id., citing Petroll W M, et al., ZO-1 reorganization and myofibroblast transformation of corneal endothelial cells after freeze injury in the cat. Exp. Eye Res. (1997) 64: 257-267). Inducers of EnMT and fibrotic changes in the endothelial layer include FGF-2, which may come from PMNs migrating to the cornea during epithelial and stromal wound healing (Id., citing Lee H T, et al., FGF-2 induced by interleukin-1 beta through the action of phosphatidylinositol 3-kinase mediates endothelial mesenchymal transformation in corneal endothelial cells. J. Biol. Chem. (2004) 279: 32325-32332) or IL-1β (Id., citing Lee J G, et al., Endothelial mesenchymal transformation mediated by IL-1β-induced FGF-2 in corneal endothelial cells. Exp. Eye Res. (2012) 95: 35-39), and TGF-β (Id. citing Sumioka T, et al., Inhibitory effect of blocking TGF-β/Smad signal on injury-induced fibrosis of corneal endothelium. Mol. Vis. (2008) 14: 2272-2281). Because EnMT may lead to fibrotic complications of healing, such as the formation of retrocorneal fibrous membrane, (Id., citing Ichijima H, et al., In vivo confocal microscopic studies of endothelial wound healing in rabbit cornea. Cornea. (1993) 12: 369-378.), some ways of attenuating EMT have been proposed. These include inhibiting the expression of connexin 43 (Id., citing Nakano Y, et al., Connexin 43 knockdown accelerates wound healing but inhibits mesenchymal transition after corneal endothelial injury in vivo. Invest. Ophthalmol. Vis. Sci. (2008) 49: 93-104) and TGF-β type I receptor (Id., citing Okumura N, et al., Inhibition of TGF-β signaling enables human corneal endothelial cell expansion in vitro for use in regenerative medicine. PLoS One. (2013) 8: e58000). The latter technique also facilitates endothelial cell propagation in culture.

Migration and spreading of corneal endothelial cells during wound healing is stimulated by a number of factors. The ECM proteins fibronectin and TSP-1 were shown to facilitate cell migration (Id., citing Munjal I D, et al., Thrombospondin: biosynthesis, distribution, and changes associated with wound repair in corneal endothelium. Eur. J. Cell Biol. (1990) 52:252-263; Gundorova R A, et al., Stimulation of penetrating corneal wound healing by exogenous fibronectin. Eur. J. Ophthalmol. (1994) 4: 202-210; Blanco-Mezquita J T, et al., Role of thrombospondin-1 in repair of penetrating corneal wounds. Invest. Ophthalmol. Vis. Sci. (2013) 54: 6262-6268.). Growth factors known to promote endothelial migration and wound healing include EGF, FGF-2, IL-1β, PDGF-BB, TGF-β2, and VEGF, whereas IGF-I and IGF-II are ineffective, and IL-4 reduces migration (Id., citing Joyce N C, et al., In vitro pharmacologic separation of corneal endothelial migration and spreading responses. Invest. Ophthalmol. Vis. Sci. (1990) 31: 1816-1826; Raphael B, et al., Enhanced healing of cat corneal endothelial wounds by epidermal growth factor. Invest. Ophthalmol. Vis. Sci. (1993) 34: 2305-2312; Soltau J B, McLaughlin B J. Effects of growth factors on wound healing in serum-deprived kitten corneal endothelial cell cultures. Cornea. (1993) 12: 208-215; Hoppenreijs V P, et al., Basic fibroblast growth factor stimulates corneal endothelial cell growth and endothelial wound healing of human corneas. Invest. Ophthalmol. Vis. Sci. (1994) 35: 931-944; Hoppenreijs V P, et al., Effects of platelet-derived growth factor on endothelial wound healing of human corneas. Invest. Ophthalmol. Vis. Sci. (1994) 35: 150-161; Hoppenreijs V P, et al., Corneal endothelium and growth factors. Sury Ophthalmol. (1996) 41: 155-164; Bednarz J, et al., Influence of vascular endothelial growth factor on bovine corneal endothelial cells in a wound-healing model. Ger. J. Ophthalmol. (1996) 5: 127-31; Sabatier P, et al., Effects of human recombinant basic fibroblast growth factor on endothelial wound healing in organ culture of human cornea. J. Fr. Ophtalmol. (1996) 19: 200-207; Thalmann-Goetsch A, et al., Comparative study on the effects of different growth factors on migration of bovine corneal endothelial cells during wound healing. Acta. Ophthalmol. Scand. (1997) 75: 490-495; Rieck P W, et al., Intracellular signaling pathway of FGF-2-modulated corneal endothelial cell migration during wound healing in vitro. Exp. Eye Res. (2001) 73: 639-650; Imanishi J, et al., Growth factors: importance in wound healing and maintenance of transparency of the cornea. Prog Retin Eye Res. (2000) 19: 113-129; Baldwin H C, Marshall J. Growth factors in corneal wound healing following refractive surgery: A review. Acta. Ophthalmol. Scand. (2002) 80: 238-247; Lee J G, Heur M. Interleukin-1β enhances cell migration through AP-1 and NF-κB pathway-dependent FGF2 expression in human corneal endothelial cells. Biol. Cell. (2013) 105: 175-189; Lee J G, Heur M. Interleukin-1β-induced Wnt5a enhances human corneal endothelial cell migration through regulation of Cdc42 and RhoA. Mol. Cell Biol. (2014) 34: 3535-3545).

The signaling pathways downstream of these factors that are important for wound healing are diverse. Prostaglandin E2 acting through cAMP pathway, ERK1/2 and p38 MAP kinase have been shown to participate in endothelial migration and wound healing (Id., citing Joyce N C, Meklir B. PGE2: a mediator of corneal endothelial wound repair in vitro. Am. J. Physiol. (1994) 266: C269-275; Sumioka T, et al., Inhibitory effect of blocking TGF-β/Smad signal on injury-induced fibrosis of corneal endothelium. Mol. Vis. (2008) 14: 2272-2281; Chen W L, et al., ERK1/2 activation regulates the wound healing process of rabbit corneal endothelial cells. Curr. Eye Res. (2009) 34: 103-111; Joko T, et al., Involvement of P38MAPK in human corneal endothelial cell migration induced by TGF-β2. Exp. Eye Res. (2013) 108: 23-32). FGF-2 stimulates migration through several pathways including p38, PI3K/Akt, and protein kinase C/phospholipase A2 (Id., citing Rieck P W, et al., Intracellular signaling pathway of FGF-2-modulated corneal endothelial cell migration during wound healing in vitro. Exp. Eye Res. (2001) 73: 639-650; L Lee H T, et al., FGF-2 induced by interleukin-1 beta (IL-1β) through the action of phosphatidylinositol 3-kinase mediates endothelial mesenchymal transformation in corneal endothelial cells. J. Biol. Chem. (2004) 279: 32325-32332; Joko T, et al., Involvement of P38MAPK in human corneal endothelial cell migration induced by TGF-β2. Exp. Eye Res. (2013) 108: 23-32). IL-1β stimulates migration through induction of FGF-2 (Id., citing Lee J G, et al., Endothelial mesenchymal transformation mediated by IL-1β-induced FGF-2 in corneal endothelial cells. Exp. Eye Res. (2012) 95: 35-39), as well as induction of Wnt5a that activate Cdc42 and inactivate RhoA (Id., citing eLe J G, Kay E P. FGF-2-induced wound healing in corneal endothelial cells requires Cdc42 activation and Rho inactivation through the phosphatidylinositol 3-kinase pathway. Invest. Ophthalmol. Vis. Sci. (2006) 47:1376-1386; Lee J G, Heur M. Interleukin-1β enhances cell migration through activator protein 1 (AP-1) and NF-κB pathway-dependent FGF2 expression in human corneal endothelial cells. Biol. Cell. (2013) 105:175-189 Lee J G, Heur M. Interleukin-1β-induced Wnt5a enhances human corneal endothelial cell migration through regulation of Cdc42 and RhoA. Mol. Cell Biol. (2014) 34: 3535-3545). In the endothelial cells, interleukin-1β stimulates cell migration directly and indirectly.

Surgical Correction of Corneal Damage

A diseased cornea may be replaced surgically with a clear, healthy cornea from a human donor (corneal transplantation) by a number of methods.

Phototherapeutic keratectomy (“PTK”) is a type of laser eye surgery that is used to treat corneal dystrophies, corneal scars, and some corneal infections. The surgeon uses a laser to remove thin layers of diseased cornea tissue microscopically, allowing new tissue to grow on the smooth surface.

If the front and middle layers of the cornea are damaged, a deep anterior lamellar keratoplasty (DALK) or partial thickness corneal transplant is performed; only the front and middle layers of the cornea are removed, with the endothelial layer kept in place. Healing time after DALK is shorter than after a full corneal transplant. There is also less risk of having the new cornea rejected. DALK is commonly used to treat keratoconus or bulging of the cornea.

If both front and inner corneal layers are damaged, penetrating keratoplasty (PK) or full thickness corneal transplant is performed to remove and replace the damaged cornea. PK has a longer recovery period than other types of corneal transplants. Getting complete vision back after PK may take up to 1 year or longer. With a PK, there is a slightly higher risk than with other types of corneal transplants that the cornea will be rejected.

In some eye conditions, the endothelium, the innermost layer of the cornea, is damaged. Endothelial keratoplasty is a surgery to replace this layer of the cornea with healthy donor tissue. It is known as a partial transplant since only the endothelium is replaced. Examples of types of endothelial kertoplasty include DSEK (or DSAEK)—Descemet's Stripping (Automated) Endothelial Keratoplasty, and DMEK—Descemet's Membrane Endothelial Keratoplasty. Each procedure removes damaged cells from Descemet's membrane by removing the damaged corneal layer through a small incision, and putting the new tissue in place. Much of the cornea is left untouched.

Optics of the Eye

Focusing the eye contracts the ciliary muscle to reduce the tension or stress transferred to the lens by the suspensory ligaments. This results in increased convexity of the lens and, thus, increases the optical power of the eye. The term “accommodation” refers to the increase in thickness and convexity of the eye's lens in response to ciliary muscle contraction in order to focus the image of an external object on the retina. The term “amplitude of accommodation” refers to the difference in refractivity of the eye at rest and when fully accommodated.

The refractive power of the human eye is measured in diopters, which is a unit of measurement of the optical power of a lens and is equal to the reciprocal of the focal length measured in meters. In humans, the total refractive power (optical power) of the relaxed eye is approximately 60 diopters. The cornea accounts for approximately two-thirds of the refractive power (i.e., 40 diopters) and the lens accounts for the remaining one-third of the refractive power (i.e., 20 diopters). (Najjar, Dany MD. “Clinical Optics and Refraction,” https://web.archive.org/web/20080323035251/http://www.eyeweb.org/optics.htm).

Emmetropia refers to an eye that has no visual defects. It is the state of vision where a faraway object at a distance of infinity is in sharp focus with the eye lens in a neutral or relaxed state. An emmetropic eye does not require vision correction.

Visual Abnormalities of the Human Eye

Abnormalities in the human eye can lead to vision impairment such as myopia (near-sightedness), hyperopia (farsightedness), astigmatism, and presbyopia.

Myopia (or nearsightedness) occurs when the human eye is too long, relative to the focusing power of the cornea and the lens of the eye. This causes light rays to focus at a point in front of the retina, rather than directly on its surface. Hyperopia (or farsightedness) occurs when light rays entering the eye focus behind the retina, rather than directly on it. Astigmatism is a vision condition that causes blurred vision and occurs when the cornea is irregularly shaped. This prevents light rays from focusing properly on the retina.

Presbyopia, as seen in FIG. 3, is generally characterized by a decrease in the eye's ability to increase its power to focus on nearby objects due to, for example, a loss of elasticity in the crystalline lens that occurs over time. Ophthalmic devices and/or procedures (e.g., contact lenses, intraocular lenses, LASIK, inlays) can be used to address presbyopia using three common approaches. With a monovision prescription, the diopter power of one eye is adjusted to focus distant objects and the power of the second eye is adjusted to focus near objects. The appropriate eye is used to clearly view the object of interest. In the next two approaches, multifocal or bifocal optics are used to simultaneously, in one eye, provide powers to focus both distant and near objects. One common multifocal design includes a central zone of higher diopter power to focus near objects, surrounded by a peripheral zone of the desired lower power to focus distant objects. In a modified monovision prescription, the diopter power of one eye is adjusted to focus distance objects, and in the second eye a multifocal optical design is induced by the intracorneal inlay. The subject therefore has the necessary diopter power from both eyes to view distant objects, while the near power zone of the multifocal eye provides the necessary power for viewing near objects. In a bilateral multifocal prescription, the multifocal optical design is induced in both eyes. Both eyes therefore contribute to both distance and near vision.

Corneal Inlay Structure and Function

A variety of devices and procedures have been developed to attempt to provide vision correction.

Laser-assisted in situ keratomileusis (“LASIK”) is a type of refractive laser eye surgery in which a laser is used to remodel a portion of the cornea after lifting a previous cut corneal flap.

A corneal inlay is an implant that is surgically inserted within the cornea beneath a portion of corneal tissue. It can be positioned by, for example, cutting a flap in the cornea and positioning the inlay beneath the flap. The corneal flap is created by making an incision in the corneal tissue and separating the corneal tissue from the underlying stroma, with one segment remaining attached, which acts like a hinge. The corneal inlay can also be positioned within a pocket (meaning a sac-like cavity) formed in the cornea. Corneal inlays can alter the refractive power of the cornea by changing the shape of the anterior surface of the cornea, by creating an optical interface between the cornea and an implant by having an index of refraction; different from that of the cornea (i.e., has intrinsic power), or both. The cornea is the strongest refracting optical element in the eye, and altering the shape of the anterior surface of the cornea can therefore be a particularly useful method for correcting vision impairments caused by refractive errors.

Corneal Inlay Procedures

Regardless of the vision correction procedure and/or devices implanted, it is important to understand the cornea's natural response to the procedure to understand how the cornea will attempt to reduce or minimize the impact of the vision correction procedure.

In a simple biomechanical model proposed by Watsky et al., Investigative Ophthalmology and Visual Science, vol. 26, pp. 240-243 (1985) (“Watsky model”), the anterior corneal surface radius of curvature is assumed to be equal to the thickness of the lamellar corneal material (i.e., flap) between the anterior corneal surface and the anterior surface of a corneal inlay plus the radius of curvature of the anterior surface of the inlay. Reviews of clinical outcomes for implanted inlays or methods for design generally discuss relatively thick inlays (e.g., greater than 200 microns thick) for which the Watsky simple biomechanical response model has some validity, because the physical size of the inlay dominates the biomechanical response of the cornea and dictates the primary anterior surface change.

When an inlay is relatively small and thin, however, the material properties of the cornea contribute significantly to the resulting change in the anterior corneal surface. Petroll et al. reported that implantation of inlays induced a thinning of the central corneal epithelium overlying the inlay. “Confocal assessment of the corneal response to intracorneal lens insertion and laser in situ keratomileusis with flap creation using IntraLase,” J. Cataract Refract. Surg., vol. 32, pp 1119-1128 (July 2006).

Huang et al. reported central epithelial thickening after myopic ablation procedures and peripheral epithelial thickening and central epithelial thinning after hyperopic ablation procedures. “Mathematical Model of Corneal Surface Smoothing After Laser Refractive Surgery,” America Journal of Ophthalmology, March 2003, pp 267-278. The theory in Huang does not address correcting for presbyopia, nor does it accurately predict changes to the anterior surface, which create a center near portion of the cornea for near vision while allowing distance vision in an area of the cornea peripheral to the center near portion. Additionally, Huang reports on removing cornea tissue by ablation as opposed to adding material to the cornea, such as an intracorneal inlay.

An understanding of the cornea's response to the correction of presbyopia, using, for example, a corneal inlay, allows the response to be compensated for when performing the procedure on the cornea.

Corneal Haze

Corneal implants can lead to the development of a cloudy or opaque appearance of the cornea, which can cause blurry vision or glare by clouding the cornea or by changing the focusing power of the eye. The impact of this corneal haze on a patient's vision is dependent on the severity of the haze and its location in the cornea. Although steroid eye drops are commonly used to treat corneal haze, in cases where the steroid eye drops are ineffective, the corneal implant is commonly removed.

The present disclosure provides a corneal implant device designed to treat presbyopia and other vision conditions using a corneal inlay. The described invention comprises a droplet molding with a high water content, which can decrease/eliminate the risk of a patient developing corneal haze. In turn, the described invention may elicit a decrease in hospital visits, and reduce patient spending.

### SUMMARY OF THE INVENTION

According to one aspect, the described invention provides a method of treating presbyopia comprising placing in a cornea of a mammalian subject a corneal inlay device of high water content the corneal inlay device comprising a thickness, a diameter, a flat or flat-like base and a dome or droplet shaped top, the dome or droplet shaped top forming a contact angle with the base, wherein the corneal inlay device, when placed in the cornea is effective: to alter a shape of the anterior surface of a cornea, and to increase an eye's ability to increase its power to focus on nearby objects, with a reduced risk of development of corneal haze compared to a control. According to one embodiment, the placing of the corneal inlay device is by cutting a flap in the cornea and positioning the inlay beneath the flap. According to another embodiment, the placing of the corneal inlay device is by positioning the inlay device within a pocket formed in the cornea. According to another embodiment, the placing of the corneal inlay device is in the cornea at a depth of about 100 microns to about 200 microns, inclusive. According to another embodiment, the placing of the corneal inlay device is in the cornea at a depth of about 130 microns to about 160 microns, inclusive. According to another embodiment, the contact angle is between 1° and 180°. According to another embodiment, the thickness of the corneal inlay ranges from at least 25 microns, at least 26 microns, at least 27 microns, at least 28 microns, at least 29 microns, at least 30 microns, at least 31 microns, at least 32 microns, at least 33 microns, at least 34 microns, at least 35 microns, at least 36 microns, at least 37 microns, at least 38 microns, at least 39 microns, at least 40 microns, at least 41 microns, at least 42 microns, at least 43 microns, at least 44 microns, at least 45 microns, at least 46 microns, at least 47 microns, at least 48 microns, at least 49 microns, at least 50 microns, at least 51 microns, at least 52 microns, at least 53 microns, at least 54 microns, at least 55 microns, at least 56 microns, at least 57 microns, at least 58 microns, at least 59 microns, to 60 microns. According to another embodiment, the thickness of the corneal inlay ranges from at least 32 microns, at least 33 microns, at least 34 microns, at least 35 microns, at least 36 microns, at least 37 microns, at least 38 microns, at least 39 microns, at least 40 microns, at least 41 microns, at least 42 microns, at least 43 microns, at least 44 microns, at least 45 microns, at least 46 microns, at least 47 microns, at least 48 microns, at least 49 microns, to 50 microns.] According to another embodiment, diameter of the corneal inlay device is at least 1 mm, at least 1.1 mm, at least 1.2 mm, at least 1.3 mm, at least 1.4 mm, at least 1.5 mm, at least 1.6 mm, at least 1.7 mm, at least 1.8 mm, at least 1.9 mm, at least 2.0 mm, at least 2.1 mm, at least 2.2 mm, at least 2.3 mm, at least 2.4 mm, at least 2.5 mm, at least 2.6 mm, at least 2.7 mm, at least 2.8 mm, at least 2.9 mm, or at least 3.0 mm. According to another embodiment, the corneal inlay device comprises water, a hydrophilic polymer, and a protein. According to another embodiment, the protein is an isolated protein, a recombinant protein, a synthetic protein, or a peptidomimetic. According to another embodiment, the hydrophilic polymer comprises polyethylene glycol (“PEG”), poly(2-methacryloyloxyethyl phosphorylcholine) (MPC), or both. According to another embodiment, water content of the corneal inlay is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%. According to another embodiment, the corneal inlay device is optically transparent, biocompatible, permeable and refractive.

The described invention also provides use of a corneal inlay device with high water content to treat presbyopia in a mammalian subject, the corneal device comprising a thickness, a diameter, a flat or flat-like base and a dome or droplet shaped top, the dome or droplet shaped top forming a contact angle with the base, wherein the inlay device when placed in the cornea is effective to alter a shape of the anterior surface of a cornea and to increase an eye's ability to increase its power to focus on nearby objects with a reduced risk of development of corneal haze, compared to a control. According to one embodiment, the placing of the corneal inlay device is by cutting a flap in the cornea and positioning the inlay beneath the flap. According to another embodiment, the placing of the corneal inlay device is by positioning the inlay device within a pocket formed in the cornea. According to one embodiment, the placing of the corneal inlay device is in the cornea at a depth of about 100 microns to about 200 microns, inclusive. According to one embodiment, the placing of the corneal inlay device is in the cornea at a depth of about 130 microns to about 160 microns, inclusive. According to one embodiment, the contact angle is between 1° and 180°. According to one embodiment, the thickness of the corneal inlay ranges from at least 25 microns, at least 26 microns, at least 27 microns, at least 28 microns, at least 29 microns, at least 30 microns, at least 31 microns, at least 32 microns, at least 33 microns, at least 34 microns, at least 35 microns, at least 36 microns, at least 37 microns, at least 38 microns, at least 39 microns, at least 40 microns, at least 41 microns, at least 42 microns, at least 43 microns, at least 44 microns, at least 45 microns, at least 46 microns, at least 47 microns, at least 48 microns, at least 49 microns, at least 50 microns, at least 51 microns, at least 52 microns, at least 53 microns, at least 54 microns, at least 55 microns, at least 56 microns, at least 57 microns, at least 58 microns, at least 59 microns, to 60 microns. According to one embodiment, the thickness of the corneal inlay range from 32 microns to 50 microns, inclusive, i.e., at least 32 microns, at least 33 microns, at least 34 microns, at least 35 microns, at least 36 microns, at least 37 microns, at least 38 microns, at least 39 microns, at least 40 microns, at least 41 microns, at least 42 microns, at least 43 microns, at least 44 microns, at least 45 microns, at least 46 microns, at least 47 microns, at least 48 microns, at least 49 microns, or 50 microns. According to one embodiment, diameter of the corneal inlay device is at least 1 mm, at least 1.1 mm, at least 1.2 mm, at least 1.3 mm, at least 1.4 mm, at least 1.5 mm, at least 1.6 mm, at least 1.7 mm, at least 1.8 mm, at least 1.9 mm, at least 2.0 mm, at least 2.1 mm, at least 2.2 mm, at least 2.3 mm, at least 2.4 mm, at least 2.5 mm, at least 2.6 mm, at least 2.7 mm, at least 2.8 mm, at least 2.9 mm, or at least 3.0 mm. According to one embodiment, the corneal inlay device comprises water, a hydrophilic polymer, and a protein. According to another embodiment, the protein is an isolated protein, a recombinant protein, a synthetic protein, or a peptidomimetic. According to one embodiment, the hydrophilic polymer comprises polyethylene glycol (“PEG”), poly(2-methacryloyloxyethyl phosphorylcholine) (MPC), or both. According to one embodiment, the water content of the corneal inlay is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%. According to one embodiment, the corneal inlay device is optically transparent, biocompatible, permeable and refractive.

These and other advantages of the invention will be apparent to those of ordinary skill in the art by reference to the following detailed description and the accompanying drawings.

In the various views of the drawings, like reference characters designate like or similar parts.

### BRIEF DESCRIPTION OF THE DRAWINGS

FIG. 1 shows an illustrative view of the human eye; (from Allaboutvision.com/resources/anatomy.htm, Accessed March 2019);

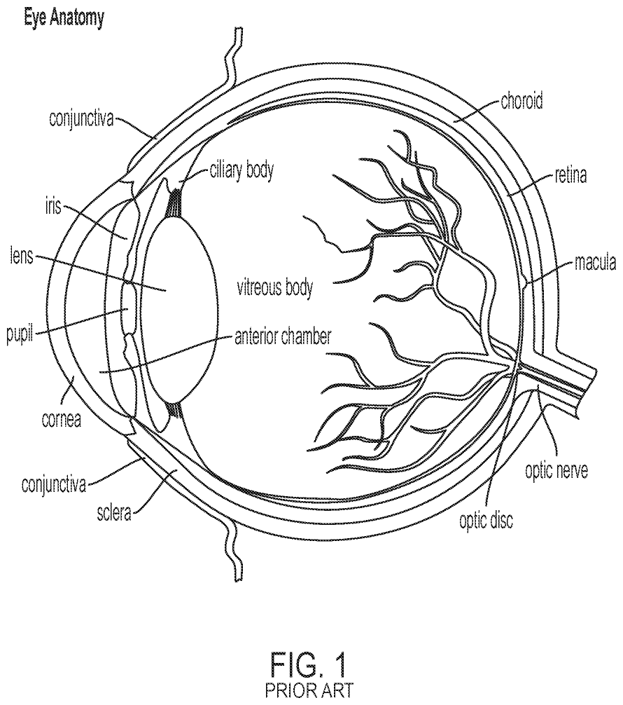

FIG. 2 shows an illustrative view of the five layers of the cornea;

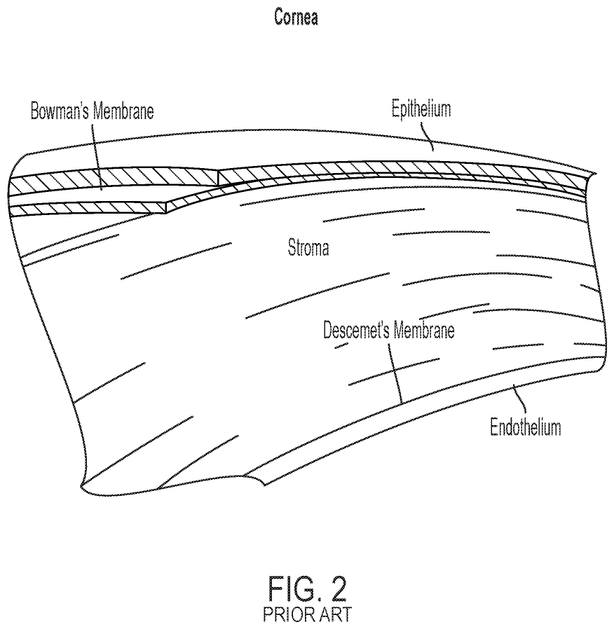

FIG. 3 shows an illustrative view of the effects of presbyopia on the human eye;

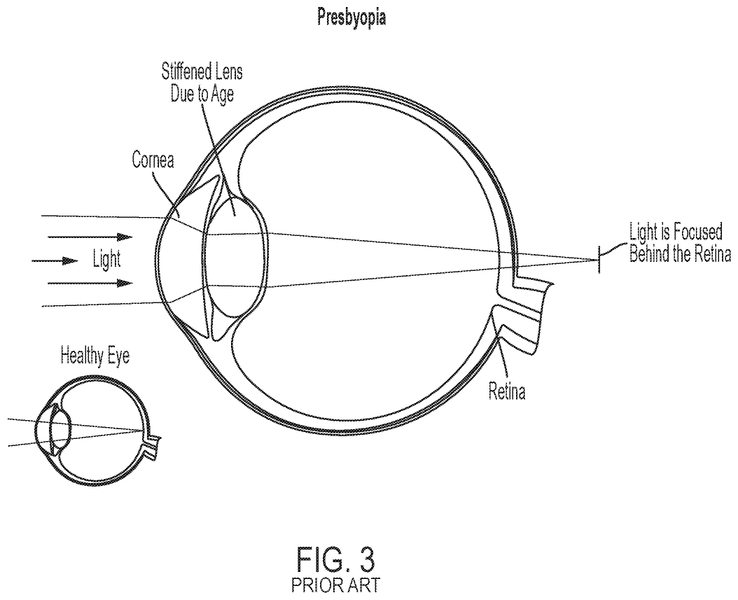

FIG. 4 shows an illustrative embodiment of the corneal inlay device of the present disclosure;

FIGS. 5A, 5B and 5C show a droplet top of the corneal inlay forming a contact angle with a base of the corneal inlay;

FIG. 6 is a diagram showing the corneal inlay of the present disclosure implanted in a cornea;

FIG. 7 shows an example of how a corneal inlay can provide near vision to a subject's eye while retaining some distance vision according to an embodiment of the present disclosure;

FIG. 8 is a graph showing a change in anterior corneal surface height and the corresponding induced added power.

FIG. 9 is a diagram showing a preoperative optical coherence tomography (“OCT”) and a postoperative OCT including an example location for the corneal inlay of the present disclosure;

FIG. 10 is a graph showing the refractive effect of water content to an inlay index of refraction and to an intrinsic power.

### DETAILED DESCRIPTION OF THE INVENTION

### Glossary

### Anatomical Terms

When referring to animals, that typically have one end with a head and mouth, with the opposite end often having the anus and tail, the head end is referred to as the cranial end, while the tail end is referred to as the caudal end. Within the head itself, rostral refers to the direction toward the end of the nose, and caudal is used to refer to the tail direction. The surface or side of an animal's body that is normally oriented upwards, away from the pull of gravity, is the dorsal side; the opposite side, typically the one closest to the ground when walking on all legs, swimming or flying, is the ventral side. On the limbs or other appendages, a point closer to the main body is “proximal”; a point farther away is “distal”. Three basic reference planes are used in zoological anatomy. A “sagittal” plane divides the body into left and right portions. The “midsagittal” plane is in the midline, i.e. it would pass through midline structures such as the spine, and all other sagittal planes are parallel to it. A “coronal” plane divides the body into dorsal and ventral portions. A “transverse” plane divides the body into cranial and caudal portions.

When referring to humans, the body and its parts are always described using the assumption that the body is standing upright. Portions of the body which are closer to the head end are “superior” (corresponding to cranial in animals), while those farther away are “inferior” (corresponding to caudal in animals). Objects near the front of the body are referred to as “anterior” (corresponding to ventral in animals); those near the rear of the body are referred to as “posterior” (corresponding to dorsal in animals). A transverse, axial, or horizontal plane is an X-Y plane, parallel to the ground, which separates the superior/head from the inferior/feet. A coronal or frontal plane is an Y-Z plane, perpendicular to the ground, which separates the anterior from the posterior. A sagittal plane is an X-Z plane, perpendicular to the ground and to the coronal plane, which separates left from right. The midsagittal plane is the specific sagittal plane that is exactly in the middle of the body.

Structures near the midline are called medial and those near the sides of animals are called lateral. Therefore, medial structures are closer to the midsagittal plane, lateral structures are further from the midsagittal plane. Structures in the midline of the body are median. For example, the tip of a human subject's nose is in the median line.

The term “ipsilateral” as used herein means on the same side, the term “contralateral” as used herein means on the other side, and the term “bilateral” as used herein means on both sides. Structures that are close to the center of the body are proximal or central, while ones more distant are distal or peripheral. For example, the hands are at the distal end of the arms, while the shoulders are at the proximal ends.

The term “biocompatible” as used herein, means causing no clinically relevant tissue irritation, injury, toxic reaction, or immunologic reaction to human tissue based on a clinical risk/benefit assessment.

The term “collagen” as used herein refers to a natural, chemically synthesized, or synthetic protein rich in glycine and proline that in vivo is a major component of the extracellular matrix and connective tissues.

The term “contact angle” as used herein refers to the angle that a liquid creates with a solid surface or capillary walls of a porous material when both materials come into contact. It is determined by properties of the solid and the liquid, the interaction and repulsion forces between liquid and solid, and by the three phase interface properties (gas, liquid and solid). The balance between the cohesive forces of similar molecules such as between the liquid molecules (i.e. hydrogen bonds and Van der Waals forces) and the adhesive forces between dissimilar molecules such as between the liquid and solid molecules (i.e. mechanical and electrostatic forces) will determine the contact angle created in the solid and liquid interface. Contact angle is a common way to measure the wettability of a surface or material. https://chem.libretexts.org/Bookshelves/Physical_and_Theoretical_Chemistry_Textbook_Maps/Supplemental_Modules_(Physical_and_Theoretical_Chemistry)/Physical_Properties_of_Matter/States_of_Matter/Properties_of_Liquids/Contact_Angle s, visited 5/17/19)

The term “corneal apex” as used herein refers to the point of maximum curvature.

The term “corneal vertex” as used herein refers to the point located at the intersection of an individual's line of fixation and the corneal surface.

The term “curvature” as used herein refers to a degree of curving of a continuously bending line, without angles.

The term “demolding” as used herein refers to a process of removing a mold from a model or a casting from a mold. The process can be, for example, by mechanical means, by hand, by the use of compressed air, etc.

The term “elasticity” as used herein refers to a measure of the deformation of an object when a force is applied. Objects that are very elastic like rubber have high elasticity and stretch easily.

The term “focal length” of a lens as used herein refers to the distance at which a lens focuses parallel rays of light. Given its diopter, the focal length of a lens can be calculated from the equation: focal length in mm=1000/diopter.

The term “hydrogel” as used herein refers to a substance resulting in a solid, semisolid, pseudoplastic, or plastic structure containing a necessary aqueous component to produce a gelatinous or jelly-like mass.

The term “hydrophilic” as used herein refers to a material or substance having an affinity for polar substances, such as water.

The term “index of refraction” as used herein refers to a measure of the extent to which a substance/medium slows down light waves passing through it. Its value determines the extent to which light is refracted (bent) when entering or leaving the substance/medium. It is the ratio of the velocity of light in a vacuum to its speed in a substance or medium.

The term “isolated” as used herein refers to material, such as, but not limited to, a nucleic acid, peptide, polypeptide, or protein, which is: (1) substantially or essentially free from components that normally accompany or interact with it as found in its naturally occurring environment. The terms “substantially free” or “essentially free” are used herein to refer to considerably or significantly free of, or more than about 95% free of, more than about 96% free of, more than about 97% free of, more than about 98% free of, or more than about 99% free of. The isolated material optionally comprises material not found with the material in its natural environment; or (2) the material has been synthetically (non-naturally) altered by deliberate human intervention.

The term “matrix” as used herein refers to a three dimensional network of fibers that contains voids (or “pores”) where the woven fibers intersect. The structural parameters of the pores, including the pore size, porosity, pore interconnectivity/tortuosity and surface area, can affect how substances (e.g., fluid, solutes) move in and out of the matrix.

The term “miosis” as used herein means excessive constriction (shrinking) of the pupil. In miosis, the diameter of the pupil is less than 2 millimeters (mm),

The term “permeable” as used herein means permitting the passage of substances, such as oxygen, glucose, water and ions, as through a membrane or other structure.

The term “protein” is used herein to refer to a large complex molecule or polypeptide composed of amino acids. The sequence of the amino acids in the protein is determined by the sequence of the bases in the nucleic acid sequence that encodes it.

The term “peptide” as used herein refers to a molecule of two or more amino acid chemically linked together. A peptide may refer to a polypeptide, protein or peptidomimetic.

The term “peptidomimetic” refers to a small protein-like chain designed to mimic or imitate a peptide. A peptidomimetic may comprise non-peptidic structural elements capable of mimicking (meaning imitating) or antagonizing (meaning neutralizing or counteracting) the biological action(s) of a natural parent peptide.

The terms “polypeptide” and “protein” are used herein in their broadest sense to refer to a sequence of subunit amino acids, amino acid analogs, or peptidomimetics. The subunits are linked by peptide bonds, except where noted. The polypeptides described herein may be chemically synthesized or recombinantly expressed. Polypeptides of the described invention are chemically synthesized. Synthetic polypeptides, prepared using the well known techniques of solid phase, liquid phase, or peptide condensation techniques, or any combination thereof, can include natural and unnatural amino acids. Amino acids used for peptide synthesis may be standard Boc (N-α-amino protected N-α-t-butyloxycarbonyl) amino acid resin with the standard deprotecting, neutralization, coupling and wash protocols of the original solid phase procedure of Merrifield (1963, J. Am. Chem. Soc. 85:2149-2154), or the base-labile N-α-amino protected 9-fluorenylmethoxycarbonyl (Fmoc) amino acids first described by Carpino and Han (1972, J. Org. Chem. 37:3403-3409). Both Fmoc and Boc N-α-amino protected amino acids can be obtained from Sigma, Cambridge Research Biochemical, or other chemical companies familiar to those skilled in the art. In addition, the polypeptides can be synthesized with other N-α-protecting groups that are familiar to those skilled in this art. Solid phase peptide synthesis may be accomplished by techniques familiar to those in the art and provided, for example, in Stewart and Young, 1984, Solid Phase Synthesis, Second Edition, Pierce Chemical Co., Rockford, Ill.; Fields and Noble, 1990, Int. J. Pept. Protein Res. 35:161-214, or using automated synthesizers. The polypeptides of the invention may comprise D-amino acids (which are resistant to L-amino acid-specific proteases in vivo), a combination of D- and L-amino acids, and various “designer” amino acids (e.g., β-methyl amino acids, C-α-methyl amino acids, and N-α-methyl amino acids, etc.) to convey special properties. Synthetic amino acids include ornithine for lysine, and norleucine for leucine or isoleucine. In addition, the polypeptides can have peptidomimetic bonds, such as ester bonds, to prepare peptides with novel properties. For example, a peptide may be generated that incorporates a reduced peptide bond, i.e., R¹—CH₂—NH—R², where R₁ and R₂ are amino acid residues or sequences. A reduced peptide bond may be introduced as a dipeptide subunit. Such a polypeptide would be resistant to protease activity, and would possess an extended half-live in vivo. Accordingly, these terms also apply to amino acid polymers in which one or more amino acid residue is an artificial chemical analogue of a corresponding naturally occurring amino acid, as well as to naturally occurring amino acid polymers. When incorporated into a protein, that protein is specifically reactive to antibodies elicited to the same protein but consisting entirely of naturally occurring amino acids. The terms “polypeptide”, “peptide” and “protein” also are inclusive of modifications including, but not limited to, glycosylation, lipid attachment, sulfation, gamma-carboxylation of glutamic acid residues, hydroxylation and ADP-ribosylation. It will be appreciated, as is well known and as noted above, that polypeptides may not be entirely linear. For instance, polypeptides may be branched as a result of ubiquitination, and they may be circular, with or without branching, generally as a result of posttranslational events, including natural processing event and events brought about by human manipulation which do not occur naturally. Circular, branched and branched circular polypeptides may be synthesized by non-translation natural process and by entirely synthetic methods, as well.

The term “polymer” as used herein refers to any of various chemical compounds made of smaller, identical molecules (called monomers) linked together. Polymers generally have high molecular weights. The incorporation of two different monomers, A and B, into a polymer chain in a statistical fashion leads to copolymers. In the limit, single monomers may alternate regularly in the chain and these are known as alternating copolymers. The monomers can be combined in a more regular fashion, either by linking extended linear sequences of one to linear sequences of the other by end-to-end addition to give block copolymers, or by attaching chains of B at points on the backbone chain of A, forming a branched structure known as a graft copolymer.

The term “recombinant DNA” refers to a DNA molecule formed by laboratory methods whereby DNA segments from different sources are joined to produce a new genetic combination.

The term “recombinant protein” as used herein refers to a protein encoded by recombinant DNA that has been cloned in a system that supports expression of the gene and translation of messenger RNA within a living cell. To make a human recombinant protein, for example, a gene of interest is isolated, cloned into an expression vector, and expressed in an expression system. Exemplary expression systems include prokaryotic organisms, as bacteria, and eukaryotic organisms, such as yeast, insect cells, plants, and mammalian cells in culture.

The term “refraction” as used herein refers to the deflection of a ray of light when it passes from one medium into another of different optical density; in passing from a denser into a rarer medium it is deflected away from a line perpendicular to the surface of the refracting medium. In passing from a rarer to a denser medium, it is bent towards this perpendicular line. The term “refraction” also refers to the act of determining the nature and degree of the refractive errors in the eye and correction of the same.

“Refractive power” of a lens as used herein refers to the reciprocal of its focal length in meters, or D=1/f, where D is the power in diopters and f is the focal length in meters.

The term “RGD motif” as used herein refers to arginylglycylaspartic acid, the binding motif of fibronectin to cell adhesion molecules, which can serve as a cell adhesion site of extracellular matrix, cell surface proteins, and integrins.

The term “shape” as used herein refers to the quality of a distinct object or body in having an external surface or outline of specific form or figure.

The terms “subject” or “individual” or “patient” are used interchangeably to refer to a member of an animal species of mammalian origin, including but not limited to, mouse, rat, cat, goat, sheep, horse, hamster, ferret, pig, dog, guinea pig, rabbit and a primate, such as, for example, a monkey, ape, or human.

The term “surface tension” as used herein refers to a property of a liquid that allows it to resist an external force due to the cohesive nature of its molecules. An attractive force exerted by the molecules of a liquid below the surface upon those at the surface-air interface, resulting from the high molecular concentration of a liquid compared to the low molecular concentration of a gas, creates an inward pull, or internal pressure, which tends to restrain the liquid from flowing.

The term “thickness” as used herein refers to a measure between opposite surfaces, from top to bottom, or in a direction perpendicular to that of the length and breadth.

The term “viscosity”, as used herein refers to the property of a fluid that resists the force tending to cause the fluid to flow. Viscosity is a measure of the fluid's resistance to flow. The resistance is caused by intermolecular friction exerted when layers of fluids attempt to slide by one another. Viscosity can be of two types: dynamic (or absolute) viscosity and kinematic viscosity. Absolute viscosity or the coefficient of absolute viscosity is a measure of the internal resistance. Dynamic (or absolute) viscosity is the tangential force per unit area required to move one horizontal plane with respect to the other at unit velocity when maintained a unit distance apart by the fluid. Dynamic viscosity is usually denoted in poise (P) or centipoise (cP), wherein 1 poise=1 g/cm², and 1 cP=0.01 P. Kinematic viscosity is the ratio of absolute or dynamic viscosity to density. Kinematic viscosity is usually denoted in Stokes (St) or Centistokes (cSt), wherein 1 St=10-4 m²/s, and 1 cSt=0.01 St.

The term “wetting” as used herein refers to how a liquid deposited on a solid (or liquid) substrate spreads out or the ability of liquids to form boundary surfaces with solid states. It is determined by measuring the contact angle that the liquid forms in contact with the solid or liquid. The smaller the contact angle or the surface tension, the larger the wetting tendency.

The term “wt %” or “weight percent” or “percent by weight” or “wt/wt %” of a component, unless specifically stated to the contrary, refers to the ratio of the weight of the component to the total weight of the composition in which the component is included, expressed as a percentage.

The term “Young's modulus” as used herein refers to a measure of elasticity, equal to the ratio of the stress acting on a substance to the strain produced. The term “stress” as used herein refers to a measure of the force put on an object over an area. The term “strain” as used herein refers to the change in length divided by the original length of the object. Change in length is proportional to the force put on it and depends on the substance from which the object is made. Change in length is proportional to the original length and inversely proportional to the cross-sectional area. Fracture is caused by a strain placed on an object such that it deforms (a change of shape) beyond its elastic limit and breaks.

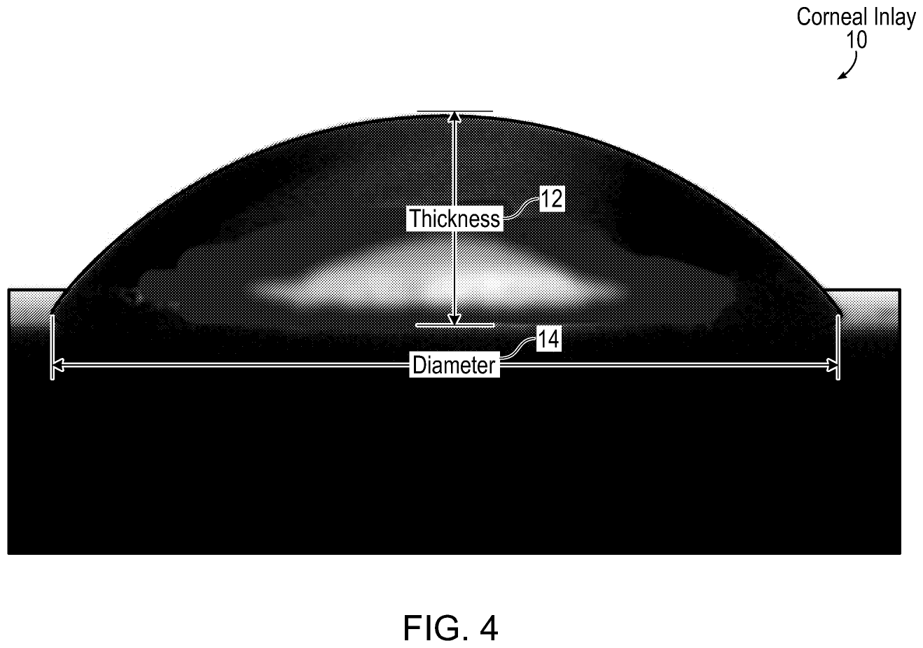

The present disclosure relates to a corneal inlay device, insertion means, and construction means, as discussed in detail below in connection with FIGS. 4-10.

FIG. 4 is a diagram showing an example of the corneal inlay 10 of the present disclosure. The corneal inlay 10 comprises a thickness 12 and a diameter 14. It can have a droplet shape, comprising a flat or flat-like base and a dome or more or less spherical, droplet shaped top. The corneal inlay 10 is biocompatible with the eye. The corneal inlay 10 comprises a diameter smaller than the diameter of the pupil and is capable of correcting presbyopia while reducing or eliminating the risk of a patient developing corneal haze. To provide near vision, the corneal inlay 10 can be implanted centrally in the cornea to induce an “effect” zone on the anterior corneal surface that is smaller than the optical zone of the cornea, wherein the “effect” zone is the area of the anterior corneal surface affected by the corneal inlay 10. The implanted corneal inlay 10 increases the curvature of the anterior corneal surface within the “effect” zone, thereby increasing the diopter power of the cornea within the “effect” zone. Because the corneal inlay 10 is smaller than the diameter of the pupil, light rays from distance objects bypass the inlay and refract through the region of the cornea peripheral to the “effect” zone to create an image of distant objects on the retina. This will be discussed in further detail below.

In exemplary embodiments, the diameter 14 of corneal inlay 10 can range from 1 millimeters (“mm”) to 3 mm, inclusive, i.e., at least 1 mm, at least 1.1 mm, at least 1.2 mm, at least 1.3 mm, at least 1.4 mm, at least 1.5 mm, at least 1.6 mm, at least 1.7 mm, at least 1.8 mm, at least 1.9 mm, at least 2.0 mm, at least 2.1 mm, at least 2.2 mm, at least 2.3 mm, at least 2.4 mm, at least 2.5 mm, at least 2.6 mm, at least 2.7 mm, at least 2.8 mm, at least 2.9 mm, or at least 3.0 mm. According to some embodiments, the diameter 14 is at least 1.0 mm. According to some embodiments, the diameter 14 is at least 1.1 mm. According to some embodiments, the diameter 14 is at least 1.2 mm. According to some embodiments, the diameter 14 is at least 1.3 mm. According to some embodiments, the diameter 14 is at least 1.4 mm. According to some embodiments, the diameter 14 is at least 1.5 mm. According to some embodiments, the diameter 14 is at least 1.6 mm. According to some embodiments, the diameter 14 is at least 1.7 mm. According to some embodiments, the diameter 14 is at least 1.8 mm. According to some embodiments, the diameter 14 is at least 1.9 mm. According to some embodiments, the diameter 14 is at least 2.0 mm. According to some embodiments, the diameter 14 is at least 2.1 mm. According to some embodiments, the diameter 14 is at least 2.2 mm. According to some embodiments, the diameter 14 is at least 2.3 mm. According to some embodiments, the diameter 14 is at least 2.4 mm. According to some embodiments, the diameter 14 is at least 2.5 mm. According to some embodiments, the diameter 14 is at least 2.6 mm. According to some embodiments, the diameter 14 is at least 2.7 mm. According to some embodiments, the diameter 14 is at least 2.8 mm. According to some embodiments, the diameter 14 is at least 2.9 mm. According to some embodiments, the diameter 14 is at least 3.0 mm.

In exemplary embodiments, the thickness 12 of corneal inlay 10 can range from 25-60 microns, inclusive, i.e., at least 25 microns, at least 26 microns, at least 27 microns, at least 28 microns, at least 29 microns, at least 30 microns, at least 31 microns, at least 32 microns, at least 33 microns, at least 34 microns, at least 35 microns, at least 36 microns, at least 37 microns, at least 38 microns, at least 39 microns, at least 40 microns, at least 41 microns, at least 42 microns, at least 43 microns, at least 44 microns, at least 45 microns, at least 46 microns, at least 47 microns, at least 48 microns, at least 49 microns, at least 50 microns, at least 51 microns, at least 52 microns, at least 53 microns, at least 54 microns, at least 55 microns, at least 56 microns, at least 57 microns, at least 58 microns, at least 59 microns, or 60 microns. According to some embodiments, the thickness 12 of corneal inlay 10 can range from 32 microns to 50 microns, inclusive, i.e., at least 32 microns, at least 33 microns, at least 34 microns, at least 35 microns, at least 36 microns, at least 37 microns, at least 38 microns, at least 39 microns, at least 40 microns, at least 41 microns, at least 42 microns, at least 43 microns, at least 44 microns, at least 45 microns, at least 46 microns, at least 47 microns, at least 48 microns, at least 49 microns, or 50 microns.]

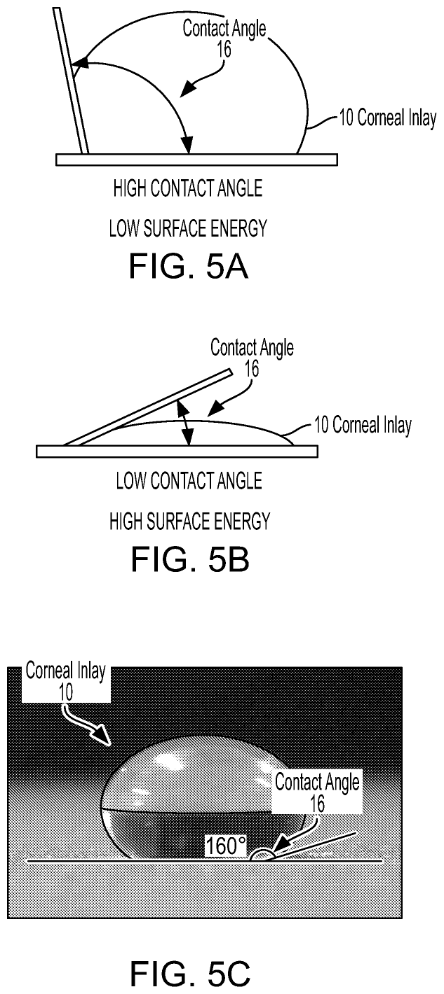

FIGS. 5A-5C show the droplet top of the corneal inlay 10 forming a contact angle 16 with the base of the corneal inlay 10. A high contact angle generates low surface energy while a low contact angle generates high surface energy. According to some embodiments, the contact angle 16 is at least 1°. According to some embodiments, the contact angle 16 is at least 2°. According to some embodiments, the contact angle 16 is at least 3°. According to some embodiments, the contact angle 16 is at least 4°. According to some embodiments, the contact angle 16 is at least 5°. According to some embodiments, the contact angle 16 is at least 6°. According to some embodiments, the contact angle 16 is at least 7°. According to some embodiments, the contact angle 16 is at least 8°. According to some embodiments, the contact angle 16 is at least 9°. According to some embodiments, the contact angle 16 is at least 10°. According to some embodiments, the contact angle 16 is at least 11°. According to some embodiments, the contact angle 16 is at least 12°. According to some embodiments, the contact angle 16 is at least 13°. According to some embodiments, the contact angle 16 is at least 14°. According to some embodiments, the contact angle 16 is at least 15°. According to some embodiments, the contact angle 16 is at least 16°. According to some embodiments, the contact angle 16 is at least 17°. According to some embodiments, the contact angle 16 is at least 18°. According to some embodiments, the contact angle 16 is at least 19°. According to some embodiments, the contact angle 16 is at least 20°. According to some embodiments, the contact angle 16 is at least 21°. According to some embodiments, the contact angle 16 is at least 22°. According to some embodiments, the contact angle 16 is at least 23°. According to some embodiments, the contact angle 16 is at least 24°. According to some embodiments, the contact angle 16 is at least 25°. According to some embodiments, the contact angle 16 is at least 26°. According to some embodiments, the contact angle 16 is at least 27°. According to some embodiments, the contact angle 16 is at least 28°. According to some embodiments, the contact angle 16 is at least 29°. According to some embodiments, the contact angle 16 is at least 30°. According to some embodiments, the contact angle 16 is at least 31°. According to some embodiments, the contact angle 16 is at least 32°. According to some embodiments, the contact angle 16 is at least 33°. According to some embodiments, the contact angle 16 is at least 34°. According to some embodiments, the contact angle 16 is at least 35°. According to some embodiments, the contact angle 16 is at least 36°. According to some embodiments, the contact angle 16 is at least 37°. According to some embodiments, the contact angle 16 is at least 38°. According to some embodiments, the contact angle 16 is at least 39°. According to some embodiments, the contact angle 16 is at least 40°. According to some embodiments, the contact angle 16 is at least 41°. According to some embodiments, the contact angle 16 is at least 42°. According to some embodiments, the contact angle 16 is at least 43°. According to some embodiments, the contact angle 16 is at least 44°. According to some embodiments, the contact angle 16 is at least 45°. According to some embodiments, the contact angle 16 is at least 46°. According to some embodiments, the contact angle 16 is at least 47°. According to some embodiments, the contact angle 16 is at least 48°. According to some embodiments, the contact angle 16 is at least 49°. According to some embodiments, the contact angle 16 is at least 50°. According to some embodiments, the contact angle 16 is at least 51°. According to some embodiments, the contact angle 16 is at least 52°. According to some embodiments, the contact angle 16 is at least 53°. According to some embodiments, the contact angle 16 is at least 54°. According to some embodiments, the contact angle 16 is at least 55°. According to some embodiments, the contact angle 16 is at least 56°. According to some embodiments, the contact angle 16 is at least 57°. According to some embodiments, the contact angle 16 is at least 58°. According to some embodiments, the contact angle 16 is at least 59°. According to some embodiments, the contact angle 16 is at least 60°. According to some embodiments, the contact angle 16 is at least 61°. According to some embodiments, the contact angle 16 is at least 62°. According to some embodiments, the contact angle 16 is at least 63°. According to some embodiments, the contact angle 16 is at least 64°. According to some embodiments, the contact angle 16 is at least 65°. According to some embodiments, the contact angle 16 is at least 66°. According to some embodiments, the contact angle 16 is at least 67°. According to some embodiments, the contact angle 16 is at least 68°. According to some embodiments, the contact angle 16 is at least 69°. According to some embodiments, the contact angle 16 is at least 70°. According to some embodiments, the contact angle 16 is at least 71°. According to some embodiments, the contact angle 16 is at least 72°. According to some embodiments, the contact angle 16 is at least 73°. According to some embodiments, the contact angle 16 is at least 74°. According to some embodiments, the contact angle 16 is at least 75°. According to some embodiments, the contact angle 16 is at least 76°. According to some embodiments, the contact angle 16 is at least 77°. According to some embodiments, the contact angle 16 is at least 78°. According to some embodiments, the contact angle 16 is at least 79°. According to some embodiments, the contact angle 16 is at least 80°. According to some embodiments, the contact angle 16 is at least 81°. According to some embodiments, the contact angle 16 is at least 82°. According to some embodiments, the contact angle 16 is at least 83°. According to some embodiments, the contact angle 16 is at least 84°. According to some embodiments, the contact angle 16 is at least 85°. According to some embodiments, the contact angle 16 is at least 86°. According to some embodiments, the contact angle 16 is at least 87°. According to some embodiments, the contact angle 16 is at least 88°. According to some embodiments, the contact angle 16 is at least 89°. According to some embodiments, the contact angle 16 is at least 80°. According to some embodiments, the contact angle 16 is at least 91°. According to some embodiments, the contact angle 16 is at least 92°. According to some embodiments, the contact angle 16 is at least 93°. According to some embodiments, the contact angle 16 is at least 94°. According to some embodiments, the contact angle 16 is at least 95°. According to some embodiments, the contact angle 16 is at least 96°. According to some embodiments, the contact angle 16 is at least 97°. According to some embodiments, the contact angle 16 is at least 98°. According to some embodiments, the contact angle 16 is at least 99°. According to some embodiments, the contact angle 16 is at least 100°. According to some embodiments, the contact angle 16 is at least 101°. According to some embodiments, the contact angle 16 is at least 102°. According to some embodiments, the contact angle 16 is at least 103°. According to some embodiments, the contact angle 16 is at least 104°. According to some embodiments, the contact angle 16 is at least 105°. According to some embodiments, the contact angle 16 is at least 106°. According to some embodiments, the contact angle 16 is at least 107°. According to some embodiments, the contact angle 16 is at least 108°. According to some embodiments, the contact angle 16 is at least 109°. According to some embodiments, the contact angle 16 is at least 110°. According to some embodiments, the contact angle 16 is at least 111°. According to some embodiments, the contact angle 16 is at least 112°. According to some embodiments, the contact angle 16 is at least 113°. According to some embodiments, the contact angle 16 is at least 114°. According to some embodiments, the contact angle 16 is at least 115°. According to some embodiments, the contact angle 16 is at least 116°. According to some embodiments, the contact angle 16 is at least 117°. According to some embodiments, the contact angle 16 is at least 118°. According to some embodiments, the contact angle 16 is at least 119°. According to some embodiments, the contact angle 16 is at least 120°. According to some embodiments, the contact angle 16 is at least 121°. According to some embodiments, the contact angle 16 is at least 122°. According to some embodiments, the contact angle 16 is at least 123°. According to some embodiments, the contact angle 16 is at least 124°. According to some embodiments, the contact angle 16 is at least 125°. According to some embodiments, the contact angle 16 is at least 126°. According to some embodiments, the contact angle 16 is at least 127°. According to some embodiments, the contact angle 16 is at least 128°. According to some embodiments, the contact angle 16 is at least 129°. According to some embodiments, the contact angle 16 is at least 130°. According to some embodiments, the contact angle 16 is at least 131°. According to some embodiments, the contact angle 16 is at least 132°. According to some embodiments, the contact angle 16 is at least 133°. According to some embodiments, the contact angle 16 is at least 134°. According to some embodiments, the contact angle 16 is at least 135°. According to some embodiments, the contact angle 16 is at least 136°. According to some embodiments, the contact angle 16 is at least 137°. According to some embodiments, the contact angle 16 is at least 138°. According to some embodiments, the contact angle 16 is at least 139°. According to some embodiments, the contact angle 16 is at least 140°. According to some embodiments, the contact angle 16 is at least 141°. According to some embodiments, the contact angle 16 is at least 142°. According to some embodiments, the contact angle 16 is at least 143°. According to some embodiments, the contact angle 16 is at least 144°. According to some embodiments, the contact angle 16 is at least 145°. According to some embodiments, the contact angle 16 is at least 146°. According to some embodiments, the contact angle 16 is at least 147°. According to some embodiments, the contact angle 16 is at least 148°. According to some embodiments, the contact angle 16 is at least 149°. According to some embodiments, the contact angle 16 is at least 150°. According to some embodiments, the contact angle 16 is at least 151°. According to some embodiments, the contact angle 16 is at least 152°. According to some embodiments, the contact angle 16 is at least 153°. According to some embodiments, the contact angle 16 is at least 154°. According to some embodiments, the contact angle 16 is at least 155°. According to some embodiments, the contact angle 16 is at least 156°. According to some embodiments, the contact angle 16 is at least 157°. According to some embodiments, the contact angle 16 is at least 158°. According to some embodiments, the contact angle 16 is at least 159°. According to some embodiments, the contact angle 16 is at least 160°. According to some embodiments, the contact angle 16 is at least 161°. According to some embodiments, the contact angle 16 is at least 162°. According to some embodiments, the contact angle 16 is at least 163°. According to some embodiments, the contact angle 16 is at least 164°. According to some embodiments, the contact angle 16 is at least 165°. According to some embodiments, the contact angle 16 is at least 166°. According to some embodiments, the contact angle 16 is at least 167°. According to some embodiments, the contact angle 16 is at least 168°. According to some embodiments, the contact angle 16 is at least 169°. According to some embodiments, the contact angle 16 is at least 170°. According to some embodiments, the contact angle 16 is at least 171°. According to some embodiments, the contact angle 16 is at least 172°. According to some embodiments, the contact angle 16 is at least 173°. According to some embodiments, the contact angle 16 is at least 174°. According to some embodiments, the contact angle 16 is at least 175°. According to some embodiments, the contact angle 16 is at least 176°. According to some embodiments, the contact angle 16 is at least 177°. According to some embodiments, the contact angle 16 is at least 178°. According to some embodiments, the contact angle 16 is at least 179°. According to some embodiments, the contact angle 16 is at least 180°.

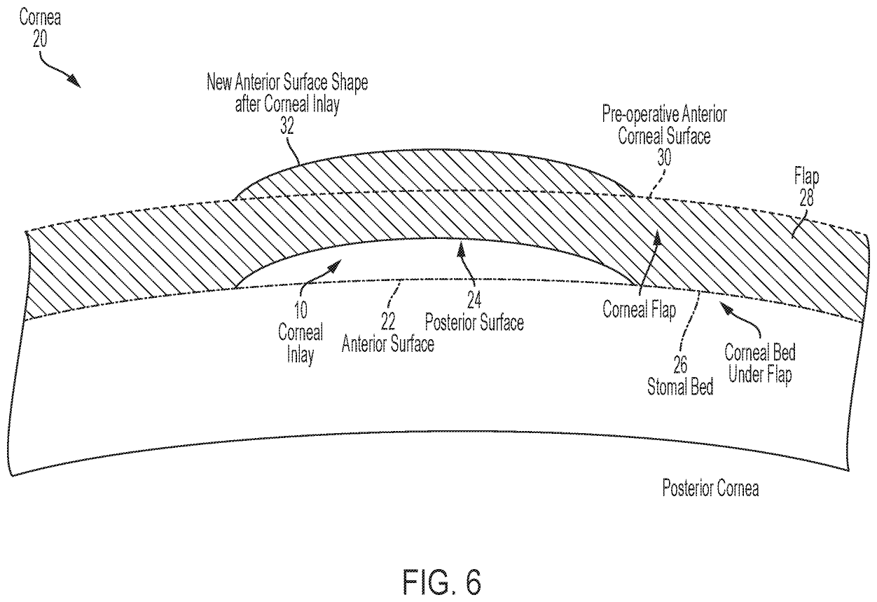

FIG. 6 is a diagram showing the corneal inlay 10 implanted in a cornea 20. The corneal inlay 10 can have a droplet shape with an anterior surface 22 and a posterior surface 24. The corneal inlay 10 can be implanted in the cornea at a depth of 50% or less of the cornea (approximately 250 μm or less), and is placed on the stromal bed 26 of the cornea 20 created by a microkeratome or any other suitable surgical instrument. For example, the corneal inlay 10 can be implanted in the cornea 20 by cutting a flap 28 into the cornea 20, lifting the flap 28 to expose an interior of the cornea 20, placing the corneal inlay 10 on the exposed area of the interior, and repositioning the flap 28 over the corneal inlay 10. The flap 28 can be cut using a laser (e.g., a femtosecond laser, a mechanical keratome, etc.) or manually by an ophthalmic surgeon. When the flap 28 is cut into the cornea 20, a small section of corneal tissue is left intact to create a hinge for the flap 28 so that the flap 28 can be repositioned accurately over the corneal inlay 10. After the flap 28 is repositioned over the corneal inlay 10, the cornea 20 heals around the flap 28 and seals the flap 28 back to the uncut peripheral portion of the anterior corneal surface. Alternatively, a pocket or well having side walls or barrier structures may be cut into the cornea 20, and the corneal inlay 10 inserted between the side walls or barrier structures through a small opening or “port” in the cornea 20.

The corneal inlay 10 changes the refractive power of the cornea by altering the shape of the anterior corneal surface. In FIG. 6, the pre-operative anterior corneal surface is represented by dashed line 30 and the post-operative anterior corneal surface induced by the underlying corneal inlay 10 is represented by solid line 32.

In some embodiments in which a corneal inlay is positioned beneath a flap, the inlay 10 is implanted between about 100 microns (micrometers) and about 200 microns deep in the cornea. In some embodiments the inlay is positioned at a depth of between about 130 microns to about 160 microns. According to some embodiments, the inlay 10 is positioned at depth of 100 microns. According to some embodiments, the inlay 10 is positioned at depth of 101 microns. According to some embodiments, the inlay 10 is positioned at depth of 102 microns. According to some embodiments, the inlay 10 is positioned at depth of 103 microns. According to some embodiments, the inlay 10 is positioned at depth of 104 microns. According to some embodiments, the inlay 10 is positioned at depth of 105 microns. According to some embodiments, the inlay 10 is positioned at depth of 106 microns. According to some embodiments, the inlay 10 is positioned at depth of 107 microns. According to some embodiments, the inlay 10 is positioned at depth of 108 microns. According to some embodiments, the inlay 10 is positioned at depth of 109 microns. According to some embodiments, the inlay 10 is positioned at depth of 110 microns. According to some embodiments, the inlay 10 is positioned at depth of 111 microns. According to some embodiments, the inlay 10 is positioned at depth of 112 microns. According to some embodiments, the inlay 10 is positioned at depth of 113 microns. According to some embodiments, the inlay 10 is positioned at depth of 114 microns. According to some embodiments, the inlay 10 is positioned at depth of 115 microns. According to some embodiments, the inlay 10 is positioned at depth of 116 microns. According to some embodiments, the inlay 10 is positioned at depth of 117 microns. According to some embodiments, the inlay 10 is positioned at depth of 118 microns. According to some embodiments, the inlay 10 is positioned at depth of 119 microns. According to some embodiments, the inlay 10 is positioned at depth of 120 microns. According to some embodiments, the inlay 10 is positioned at depth of 121 microns. According to some embodiments, the inlay 10 is positioned at depth of 122 microns. According to some embodiments, the inlay 10 is positioned at depth of 123 microns. According to some embodiments, the inlay 10 is positioned at depth of 124 microns. According to some embodiments, the inlay 10 is positioned at depth of 125 microns. According to some embodiments, the inlay 10 is positioned at depth of 126 microns. According to some embodiments, the inlay 10 is positioned at depth of 127 microns. According to some embodiments, the inlay 10 is positioned at depth of 128 microns. According to some embodiments, the inlay 10 is positioned at depth of 129 microns. According to some embodiments, the inlay 10 is positioned at depth of 130 microns. According to some embodiments, the inlay 10 is positioned at depth of 131 microns. According to some embodiments, the inlay 10 is positioned at depth of 132 microns. According to some embodiments, the inlay 10 is positioned at depth of 133 microns. According to some embodiments, the inlay 10 is positioned at depth of 134 microns. According to some embodiments, the inlay 10 is positioned at depth of 135 microns. According to some embodiments, the inlay 10 is positioned at depth of 136 microns. According to some embodiments, the inlay 10 is positioned at depth of 137 microns. According to some embodiments, the inlay 10 is positioned at depth of 138 microns. According to some embodiments, the inlay 10 is positioned at depth of 139 microns. According to some embodiments, the inlay 10 is positioned at depth of 140 microns. According to some embodiments, the inlay 10 is positioned at depth of 141 microns. According to some embodiments, the inlay 10 is positioned at depth of 142 microns. According to some embodiments, the inlay 10 is positioned at depth of 143 microns. According to some embodiments, the inlay 10 is positioned at depth of 144 microns. According to some embodiments, the inlay 10 is positioned at depth of 145 microns. According to some embodiments, the inlay 10 is positioned at depth of 146 microns. According to some embodiments, the inlay 10 is positioned at depth of 147 microns. According to some embodiments, the inlay 10 is positioned at depth of 148 microns. According to some embodiments, the inlay 10 is positioned at depth of 149 microns. According to some embodiments, the inlay 10 is positioned at depth of 150 microns. According to some embodiments, the inlay 10 is positioned at depth of 151 microns. According to some embodiments, the inlay 10 is positioned at depth of 152 microns. According to some embodiments, the inlay 10 is positioned at depth of 153 microns. According to some embodiments, the inlay 10 is positioned at depth of 154 microns. According to some embodiments, the inlay 10 is positioned at depth of 155 microns. According to some embodiments, the inlay 10 is positioned at depth of 156 microns. According to some embodiments, the inlay 10 is positioned at depth of 157 microns. According to some embodiments, the inlay 10 is positioned at depth of 158 microns. According to some embodiments, the inlay 10 is positioned at depth of 159 microns. According to some embodiments, the inlay 10 is positioned at depth of 160 microns. According to some embodiments, the inlay 10 is positioned at depth of 161 microns. According to some embodiments, the inlay 10 is positioned at depth of 162 microns. According to some embodiments, the inlay 10 is positioned at depth of 163 microns. According to some embodiments, the inlay 10 is positioned at depth of 164 microns. According to some embodiments, the inlay 10 is positioned at depth of 165 microns. According to some embodiments, the inlay 10 is positioned at depth of 166 microns. According to some embodiments, the inlay 10 is positioned at depth of 167 microns. According to some embodiments, the inlay 10 is positioned at depth of 168 microns. According to some embodiments, the inlay 10 is positioned at depth of 169 microns. According to some embodiments, the inlay 10 is positioned at depth of 170 microns. According to some embodiments, the inlay 10 is positioned at depth of 171 microns. According to some embodiments, the inlay 10 is positioned at depth of 172 microns. According to some embodiments, the inlay 10 is positioned at depth of 173 microns. According to some embodiments, the inlay 10 is positioned at depth of 174 microns. According to some embodiments, the inlay 10 is positioned at depth of 175 microns. According to some embodiments, the inlay 10 is positioned at depth of 176 microns. According to some embodiments, the inlay 10 is positioned at depth of 177 microns. According to some embodiments, the inlay 10 is positioned at depth of 178 microns. According to some embodiments, the inlay 10 is positioned at depth of 179 microns. According to some embodiments, the inlay 10 is positioned at depth of 180 microns. According to some embodiments, the inlay 10 is positioned at depth of 181 microns. According to some embodiments, the inlay 10 is positioned at depth of 182 microns. According to some embodiments, the inlay 10 is positioned at depth of 183 microns. According to some embodiments, the inlay 10 is positioned at depth of 184 microns. According to some embodiments, the inlay 10 is positioned at depth of 185 microns. According to some embodiments, the inlay 10 is positioned at depth of 186 microns. According to some embodiments, the inlay 10 is positioned at depth of 187 microns. According to some embodiments, the inlay 10 is positioned at depth of 188 microns. According to some embodiments, the inlay 10 is positioned at depth of 189 microns. According to some embodiments, the inlay 10 is positioned at depth of 190 microns. According to some embodiments, the inlay 10 is positioned at depth of 191 microns. According to some embodiments, the inlay 10 is positioned at depth of 192 microns. According to some embodiments, the inlay 10 is positioned at depth of 193 microns. According to some embodiments, the inlay 10 is positioned at depth of 194 microns. According to some embodiments, the inlay 10 is positioned at depth of 195 microns. According to some embodiments, the inlay 10 is positioned at depth of 196 microns. According to some embodiments, the inlay 10 is positioned at depth of 197 microns. According to some embodiments, the inlay 10 is positioned at depth of 198 microns. According to some embodiments, the inlay 10 is positioned at depth of 199 microns. According to some embodiments, the inlay 10 is positioned at depth of 200 microns. According to some embodiments, the depth in the cornea for a pocket may be greater than for a flap. According to some exemplary embodiments, because depth in the cornea for the pocket is greater than for the flap, a thicker inlay may be needed in order to impart a refractive correction.

The elastic (Young's) modulus of the corneal inlay 10 can, by way of example, be 0.18 megapascals (“MPa”) with a tolerance of ±0.06 MPa. However, in some embodiments, the elastic modulus of the corneal inlay 10 can exceed the tolerance. According to some embodiments, the elastic modulus of the corneal inlay 10 can be at least 0.05 MPa. According to some embodiments, the elastic modulus of the corneal inlay 10 can be at least 0.06 MPa. According to some embodiments, the elastic modulus of the corneal inlay 10 can be at least 0.07 MPa. According to some embodiments, the elastic modulus of the corneal inlay 10 can be at least 0.08 MPa. According to some embodiments, the elastic modulus of the corneal inlay 10 can be at least 0.09 MPa. According to some embodiments, the elastic modulus of the corneal inlay 10 can be at least 0.10 MPa. According to some embodiments, the elastic modulus of the corneal inlay 10 can be at least 0.11 MPa. According to some embodiments, the elastic modulus of the corneal inlay 10 can be at least 0.12 MPa. According to some embodiments, the elastic modulus of the corneal inlay 10 can be at least 0.13 MPa. According to some embodiments, the elastic modulus of the corneal inlay 10 can be at least 0.14 MPa. According to some embodiments, the elastic modulus of the corneal inlay 10 can be at least 0.15 MPa. According to some embodiments, the elastic modulus of the corneal inlay 10 can be at least 0.16 MPa. According to some embodiments, the elastic modulus of the corneal inlay 10 can be at least 0.17 MPa. According to some embodiments, the elastic modulus of the corneal inlay 10 can be at least 0.18 MPa. According to some embodiments, the elastic modulus of the corneal inlay 10 can be at least 0.19 MPa. According to some embodiments, the elastic modulus of the corneal inlay 10 can be at least 0.20 MPa. According to some embodiments, the elastic modulus of the corneal inlay 10 can be at least 0.21 MPa. According to some embodiments, the elastic modulus of the corneal inlay 10 can be at least 0.22 MPa. According to some embodiments, the elastic modulus of the corneal inlay 10 can be at least 0.23 MPa. According to some embodiments, the elastic modulus of the corneal inlay 10 can be at least 0.24 MPa. According to some embodiments, the elastic modulus of the corneal inlay 10 can be at least 0.25 MPa. According to some embodiments, the elastic modulus of the corneal inlay 10 can be at least 0.26 MPa. According to some embodiments, the elastic modulus of the corneal inlay 10 can be at least 0.27 MPa. According to some embodiments, the elastic modulus of the corneal inlay 10 can be at least 0.28 MPa. According to some embodiments, the elastic modulus of the corneal inlay 10 can be at least 0.29 MPa. According to some embodiments, the elastic modulus of the corneal inlay 10 can be at least 0.30 MPa.

The elongation at break of the corneal inlay 10 can be 58.30% with a tolerance of ±4.49%. However, in some embodiments, the elongation at break of the corneal inlay 10 can exceed the tolerance. According to some embodiments, the elongation at break of the corneal inlay 10 can be at least 48%. According to some embodiments, the elongation at break of the corneal inlay 10 can be at least 49%. According to some embodiments, the elongation at break of the corneal inlay 10 can be at least 50%. According to some embodiments, the elongation at break of the corneal inlay 10 can be at least 21%. According to some embodiments, the elongation at break of the corneal inlay 10 can be at least 52%. According to some embodiments, the elongation at break of the corneal inlay 10 can be at least 53%. According to some embodiments, the elongation at break of the corneal inlay 10 can be at least 54%. According to some embodiments, the elongation at break of the corneal inlay 10 can be at least 55%. According to some embodiments, the elongation at break of the corneal inlay 10 can be at least 56%. According to some embodiments, the elongation at break of the corneal inlay 10 can be at least 57%. According to some embodiments, the elongation at break of the corneal inlay 10 can be at least 58%. According to some embodiments, the elongation at break of the corneal inlay 10 can be at least 59%. According to some embodiments, the elongation at break of the corneal inlay 10 can be at least 60%. According to some embodiments, the elongation at break of the corneal inlay 10 can be at least 61%. According to some embodiments, the elongation at break of the corneal inlay 10 can be at least 62%. According to some embodiments, the elongation at break of the corneal inlay 10 can be at least 63%. According to some embodiments, the elongation at break of the corneal inlay 10 can be at least 64%. According to some embodiments, the elongation at break of the corneal inlay 10 can be at least 65%. According to some embodiments, the elongation at break of the corneal inlay 10 can be at least 66%. According to some embodiments, the elongation at break of the corneal inlay 10 can be at least 67%. According to some embodiments, the elongation at break of the corneal inlay 10 can be at least 68%. According to some embodiments, the elongation at break of the corneal inlay 10 can be at least 69%. According to some embodiments, the elongation at break of the corneal inlay 10 can be at least 70%.

The tensile strength (meaning the resistance of a material to breaking under tension) of the corneal inlay 10 can be 0.07 MPa with a tolerance of ±0.02 MPa. In some embodiments, the tensile strength of the corneal inlay can exceed the tolerance. According to some embodiments, the tensile strength of the corneal inlay 10 can be at least 0.01 MPa. According to some embodiments, the tensile strength of the corneal inlay 10 can be at least 0.02 MPa. According to some embodiments, the tensile strength of the corneal inlay 10 can be at least 0.03 MPa. According to some embodiments, the tensile strength of the corneal inlay 10 can be at least 0.04 MPa. According to some embodiments, the tensile strength of the corneal inlay 10 can be at least 0.05 MPa. According to some embodiments, the tensile strength of the corneal inlay 10 can be at least 0.06 MPa. According to some embodiments, the tensile strength of the corneal inlay 10 can be at least 0.07 MPa. According to some embodiments, the tensile strength of the corneal inlay 10 can be at least 0.08 MPa. According to some embodiments, the tensile strength of the corneal inlay 10 can be at least 0.09 MPa. According to some embodiments, the tensile strength of the corneal inlay 10 can be at least 0.10 MPa. According to some embodiments, the tensile strength of the corneal inlay 10 can be at least 0.11 MPa. According to some embodiments, the tensile strength of the corneal inlay 10 can be at least 0.12 MPa. According to some embodiments, the tensile strength of the corneal inlay 10 can be at least 0.13 MPa. According to some embodiments, the tensile strength of the corneal inlay 10 can be at least 0.14 MPa. According to some embodiments, the tensile strength of the corneal inlay 10 can be at least 0.15 MPa.

The backscatter (meaning deflection of radiation or particles through an angle of 180°) of the corneal inlay 10 can be 0.90% with a tolerance of ±0.17%. However, in some embodiments, the backscatter of the corneal inlay 10 can exceed the tolerance. According to some embodiments, the backscatter of the corneal inlay 10 can be at least 0.65%. According to some embodiments, the backscatter of the corneal inlay 10 can be at least 0.66%. According to some embodiments, the backscatter of the corneal inlay 10 can be at least 0.67%. According to some embodiments, the backscatter of the corneal inlay 10 can be at least 0.68%. According to some embodiments, the backscatter of the corneal inlay 10 can be at least 0.69%. According to some embodiments, the backscatter of the corneal inlay 10 can be at least 0.70%. According to some embodiments, the backscatter of the corneal inlay 10 can be at least 0.71%. According to some embodiments, the backscatter of the corneal inlay 10 can be at least 0.72%. According to some embodiments, the backscatter of the corneal inlay 10 can be at least 0.73%. According to some embodiments, the backscatter of the corneal inlay 10 can be at least 0.74%. According to some embodiments, the backscatter of the corneal inlay 10 can be at least 0.75%. According to some embodiments, the backscatter of the corneal inlay 10 can be at least 0.76%. According to some embodiments, the backscatter of the corneal inlay 10 can be at least 0.77%. According to some embodiments, the backscatter of the corneal inlay 10 can be at least 0.78%. According to some embodiments, the backscatter of the corneal inlay 10 can be at least 0.79%. According to some embodiments, the backscatter of the corneal inlay 10 can be at least 0.80%. According to some embodiments, the backscatter of the corneal inlay 10 can be at least 0.81%. According to some embodiments, the backscatter of the corneal inlay 10 can be at least 0.82%. According to some embodiments, the backscatter of the corneal inlay 10 can be at least 0.83%. According to some embodiments, the backscatter of the corneal inlay 10 can be at least 0.84%. According to some embodiments, the backscatter of the corneal inlay 10 can be at least 0.85%. According to some embodiments, the backscatter of the corneal inlay 10 can be at least 0.86%. According to some embodiments, the backscatter of the corneal inlay 10 can be at least 0.87%. According to some embodiments, the backscatter of the corneal inlay 10 can be at least 0.88%. According to some embodiments, the backscatter of the corneal inlay 10 can be at least 0.89%. According to some embodiments, the backscatter of the corneal inlay 10 can be at least 0.90%. According to some embodiments, the backscatter of the corneal inlay 10 can be at least 0.91%. According to some embodiments, the backscatter of the corneal inlay 10 can be at least 0.92%. According to some embodiments, the backscatter of the corneal inlay 10 can be at least 0.93%. According to some embodiments, the backscatter of the corneal inlay 10 can be at least 0.94%. According to some embodiments, the backscatter of the corneal inlay 10 can be at least 0.95%. According to some embodiments, the backscatter of the corneal inlay 10 can be at least 0.96%. According to some embodiments, the backscatter of the corneal inlay 10 can be at least 0.97%. According to some embodiments, the backscatter of the corneal inlay 10 can be at least 0.98%. According to some embodiments, the backscatter of the corneal inlay 10 can be at least 0.99%. According to some embodiments, the backscatter of the corneal inlay 10 can be at least 1.00%. According to some embodiments, the backscatter of the corneal inlay 10 can be at least 1.01%. According to some embodiments, the backscatter of the corneal inlay 10 can be at least 1.02%. According to some embodiments, the backscatter of the corneal inlay 10 can be at least 1.03%. According to some embodiments, the backscatter of the corneal inlay 10 can be at least 1.04%. According to some embodiments, the backscatter of the corneal inlay 10 can be at least 1.05%. According to some embodiments, the backscatter of the corneal inlay 10 can be at least 1.06%. According to some embodiments, the backscatter of the corneal inlay 10 can be at least 1.07%. According to some embodiments, the backscatter of the corneal inlay 10 can be at least 1.08%. According to some embodiments, the backscatter of the corneal inlay 10 can be at least 1.09%. According to some embodiments, the backscatter of the corneal inlay 10 can be at least 1.10%. According to some embodiments, the backscatter of the corneal inlay 10 can be at least 1.11%. According to some embodiments, the backscatter of the corneal inlay 10 can be at least 1.12%. According to some embodiments, the backscatter of the corneal inlay 10 can be at least 1.13%. According to some embodiments, the backscatter of the corneal inlay 10 can be at least 1.14%. According to some embodiments, the backscatter of the corneal inlay 10 can be at least 1.15%.

The light transmission (meaning the moving of electromagnetic waves through) of the corneal inlay 10 can be 92.4% with a tolerance of ±0.95%. In some embodiments, the elastic modulus of the corneal inlay 10 can exceed the tolerance. According to some embodiments, the light transmission of the corneal inlay 10 can be at least 85.0%. According to some embodiments, the light transmission of the corneal inlay 10 can be at least 86.0%. According to some embodiments, the light transmission of the corneal inlay 10 can be at least 87.0%. According to some embodiments, the light transmission of the corneal inlay 10 can be at least 88.0%. According to some embodiments, the light transmission of the corneal inlay 10 can be at least 89.0%. According to some embodiments, the light transmission of the corneal inlay 10 can be at least 90.0%. According to some embodiments, the light transmission of the corneal inlay 10 can be at least 91.0%. According to some embodiments, the light transmission of the corneal inlay 10 can be at least 92.0%. According to some embodiments, the light transmission of the corneal inlay 10 can be at least 93.0%. According to some embodiments, the light transmission of the corneal inlay 10 can be at least 94.0%. According to some embodiments, the light transmission of the corneal inlay 10 can be at least 95.0%. According to some embodiments, the light transmission of the corneal inlay 10 can be at least 96.0%. According to some embodiments, the light transmission of the corneal inlay 10 can be at least 97.0%. According to some embodiments, the light transmission of the corneal inlay 10 can be at least 98.0%. According to some embodiments, the light transmission of the corneal inlay 10 can be at least 99.0%. According to some embodiments, the light transmission of the corneal inlay 10 can be 100.0%.

The morphology (meaning form) of the corneal inlay 10 can be a fibrillary network with nano-pores. According to some embodiments, the nano-pores of the corneal inlay 10 can have a diameter of at least 0.1 μm. According to some embodiments, the nano-pores of the corneal inlay 10 can have a diameter of at least 0.2 μm. According to some embodiments, the nano-pores of the corneal inlay 10 can have a diameter of at least 0.3 μm. According to some embodiments, the nano-pores of the corneal inlay 10 can have a diameter of at least 0.4 μm. According to some embodiments, the nano-pores of the corneal inlay 10 can have a diameter of at least 0.5 μm. According to some embodiments, the nano-pores of the corneal inlay 10 can have a diameter of at least 0.6 μm. According to some embodiments, the nano-pores of the corneal inlay 10 can have a diameter of at least 0.7 μm. According to some embodiments, the nano-pores of the corneal inlay 10 can have a diameter of at least 0.8 μm. According to some embodiments, the nano-pores of the corneal inlay 10 can have a diameter of at least 0.9 μm. According to some embodiments, the nano-pores of the corneal inlay 10 can have a diameter of at least 1.0 μm. According to some embodiments, the nano-pores can have a diameter of approximately 0.4 μm According to some embodiments, the storage temperature for the corneal inlay 10 can range from about 2°-6° Celsius, i.e., about 2° C., 2.5° C., 3° C., 3.5° C., 4° C., 4.5° C., 5° C., 5.5° C., 6° C.

Presbyopic Inlays

According to some embodiments, the diameter of the corneal inlay 10 is small in comparison with the diameter of the pupil for correcting presbyopia. In some embodiments, a corneal inlay 10 (e.g., 1 mm to 3 mm in diameter) is implanted centrally in the cornea to induce an “effect” zone on the anterior corneal surface that is smaller than the optical zone of the cornea for providing near vision. Here, the “effect” zone is the area of the anterior corneal surface affected by the corneal inlay 10. The implanted corneal inlay 10 increases the curvature of the anterior corneal surface within the “effect” zone, thereby increasing the diopter power of the cornea within the “effect” zone. Distance vision is provided by the region of the cornea peripheral to the “effect” zone.

Presbyopia is characterized by a decrease in the ability of the eye to increase its power to focus on nearby objects due to a loss of elasticity in the crystalline lens with age. Typically, a person suffering from presbyopia requires reading glasses to provide near vision.

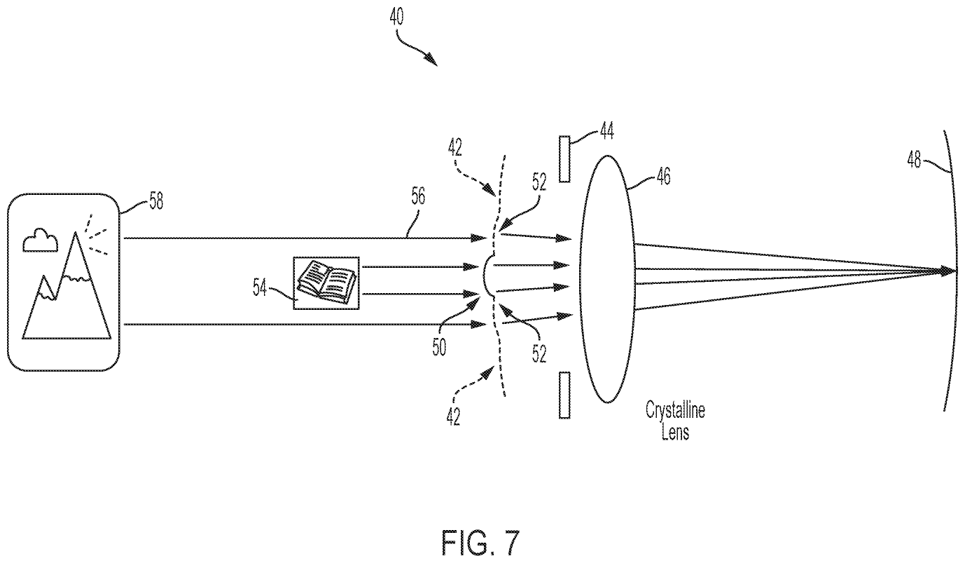

FIG. 7 shows an example of how a corneal inlay 10 can provide near vision to a subject's eye while retaining some distance vision according to an embodiment of the invention. The eye 40 comprises a cornea 42, a pupil 44, a crystalline lens 46 and a retina 48. In this example, the corneal inlay 10 (not shown) is implanted centrally in the cornea 42 to create a small diameter “effect” zone 50. The corneal inlay 10 has a smaller diameter than the pupil 44 so that the resulting “effect” zone 50 has a smaller diameter than the optical zone of the cornea 42. The “effect” zone 50 provides near vision by increasing the curvature of the anterior corneal surface, and therefore the diopter power within the “effect” zone 50. The region 52 of the cornea peripheral to the “effect” zone provides distance vision.

To increase the diopter power within the “effect” zone 50, the corneal inlay 10 has a curvature higher than the curvature of the pre-implant anterior corneal surface in order to increase the curvature of the anterior corneal surface within the “effect” zone 50. The corneal inlay 10 can further increase the diopter power within the “effect” zone 52 by having an index of refraction that is higher than the index of refraction of the cornea (n_{cornea}=1.376). Thus, the increase in the diopter power within the “effect” zone 50 can be due to the change in the anterior corneal surface induced by the corneal inlay 10 or a combination of the change in the anterior cornea surface and the index of refraction of the corneal inlay 10. For early presbyopia (e.g., about 45 to 55 years of age), at least 1 diopter is typically required for near vision. For complete presbyopia (e.g., about 60 years of age or older), between 2 and 3 diopters of additional power are required.

An advantage of corneal inlay 10 is that when concentrating on nearby objects 54, the pupil naturally becomes smaller (e.g., near point miosis) making the corneal inlay effect even more effective. Further increases in the corneal inlay effect can be achieved by increasing the illumination of a nearby object (e.g., turning up a reading light).

Because the inlay is smaller than the diameter of the pupil 44, light rays 56 from distant objects 58 bypass the inlay and refract using the region of the cornea peripheral to the “effect” zone to create an image of the distant objects on the retina 48, as shown in FIG. 7. This is particularly true with larger pupils. At night, when distance vision is most important, the pupil naturally becomes larger, thereby reducing the inlay effect and maximizing distance vision.

A subject's natural distance vision is in focus only if the subject is emmetropic (i.e., does not require glasses for distance vision). Many subjects are ammetropic, requiring either myopic or hyperopic refractive correction. Especially for myopes, distance vision correction can be provided by myopic Laser in Situ Keratomileusis (“LASIK”), Laser Epithelial Keratomileusis (“LASEK”), Photorefractive Keratectomy (“PRK”) or other similar corneal refractive procedures. After the distance corrective procedure is completed, the corneal inlay 10 can be implanted in the cornea to provide near vision. Since LASIK requires the creation of a flap, the corneal inlay 10 may be inserted concurrently with the LASIK procedure. The corneal inlay 10 can also be inserted into the cornea after the LASIK procedure since the flap can be re-opened. Therefore, the corneal inlay 10 can be used in conjunction with other refractive procedures, such as LASIK for correcting myopia or hyperopia.

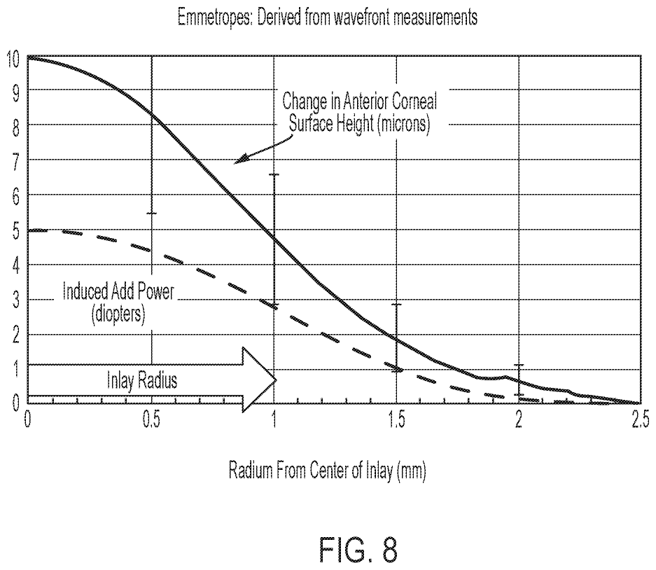

FIG. 8 is a plot of anterior corneal surface height (in microns) (y axis) vs. radius from center of inlay (mm) (x-axis). The graph shows the change in anterior corneal surface height (in microns) and the corresponding induced added power (e.g., diopters).

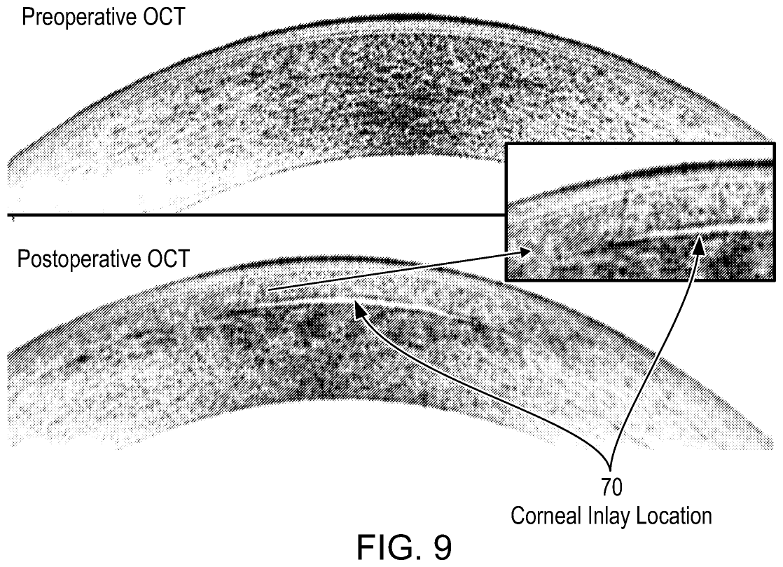

FIG. 9 is a diagram showing a preoperative optical coherence tomography (“OCT”) and a postoperative OCT. In the postoperative OCT, an example location 70 for the corneal inlay 10 is shown.

Material Chemistry of the Inlay

According to some embodiments, the inlay material comprises a biopolymer. According to some embodiments, the biopolymer is a synthetic self-assembling biopolymer. According to some embodiments, the biopolymer is a naturally-occurring biopolymer. Exemplary naturally-occurring biopolymers include, but are not limited to, protein polymers, collagen, polysaccharides, and photopolymerizable compounds. Exemplary protein polymers synthesized from self-assembling protein polymers include, for example, silk fibroin, elastin, collagen, and combinations thereof. According to some embodiments, the synthetic self-assembling biopolymer is a synthetic collagen. According to some embodiments, the collagen is a collagen mimetic peptide. As used herein, the term “mimetic” refers to chemicals containing chemical moieties that mimic the function of a peptide. For example, if a peptide contains two charged chemical moieties having functional activity, a mimetic places two charged chemical moieties in a spatial orientation and constrained structure so that the charged chemical function is maintained in three-dimensional space.

According to some embodiments, the inlay materials comprise a synthetic polymeric material. According to some embodiments the synthetic material is an optically transparent material. According to some embodiments the synthetic materials is a biocompatible material. According to some embodiments the synthetic material is a hydrophilic material. According to some embodiments the synthetic materials is a material permeable to low molecular weight nutrients so as to maintain corneal health. According to some embodiments the synthetic materials is a refractive material. According to some embodiments the synthetic material is optically transparent, biocompatible, hydrophilic, permeable and refractive.

Exemplary biocompatible biodegradable polymers include, without limitation, a poly(lactide); a poly(glycolide); a poly(lactide-co-glycolide); a poly(lactic acid); a poly(glycolic acid); a poly(lactic acid-co-glycolic acid); a poly(caprolactone); a poly(orthoester); a polyanhydride; a poly(phosphazene); a polyhydroxyalkanoate; a poly(hydroxybutyrate); a polycarbonate; a tyrosine polycarbonate; a polyamide; a polyesteramide; a polyester; a poly(dioxanone); a poly(alkylene alkylate); a polyether (such as polyethylene glycol, PEG, and polyethylene oxide, PEO); polyvinyl pyrrolidone or PVP; a polyurethane; a polyetherester; a polyacetal; a polycyanoacrylate; a poly(oxyethylene)/poly(oxypropylene) copolymer; a polyacetal, a polyketal; a polyphosphate; a (phosphorous-containing) polymer; a polyphosphoester; a polyhydroxyvalerate; a polyalkylene oxalate; a polyalkylene succinate; or a poly(maleic acid). The water-soluble, biocompatible polymer poly(2-methacryloyloxyethyl phosphorylcholine) (PMPC) is a zwitterionic polymer that is able to form a more compact conformation in aqueous solution than poly(ethylene glycol) (PEG).

Exemplary non-degradable biocompatible polymers include, without limitation, polysiloxane, polyvinyl alcohol, and polyimide,

Exemplary copolymers include, hydroxyethyl methacrylate and methyl methacrylate, and hydroxyethyl methacrylate copolymerized with polyvinyl pyrrolidone (PVP, to increase water retention) or ethylene glycol dimethacrylic acid (EGDM). Nexofilcon A (Bausch & Lomb) is a hydrophilic copolymer of 2-hydroxyethyl methacrylate and N-vinyl pyrrolidone.

Exemplary block polymers comprising blocks of hydrophilic biocompatible polymers or biopolymers or biodegradable polymers include polyethers, including polyethylene glycol, PEG; polyethylene oxide, PEO; polypropylene oxide, PPO, perfluoropolyethers (PFPEs) and block copolymers comprised of combinations thereof.

According to some embodiments, the hydrophilic polymer comprises a hydrogel polymer. Hydrogels are water-swollen, cross-linked polymeric structures produced by the polymerization reaction of one or more monomers or by association of bonds, such as hydrogen bonds and strong van der Waals interactions between chains that exist in a state between rigid solids and liquid. Aqueous gels are formed when high molecular weight polymers or high polymer concentration are incorporated in the formulations. Hydrogels generally comprise a variety of polymers. Exemplary polymers include acrylic acid, acrylamide and 2-hydroxyethylmethacrylate (HEMA). For example, Cross-linked poly (acrylic acid) of high molecular weight is commercially available as Carbopol® (B.F./Goodrich Chemical Co., Cleveland, Ohio). Polyethylene glycol diacrylate (PEGDA 400) is a long-chain, hydrophilic, crosslinking monomer. Methacryloyloxyethyl phosphorylcholine (MPC), containing a phosphorylcholine group in the side chain, is a monomer to mimic the phospholipid polar groups contained with cell membranes. Polyoxamers, commercially available as Pluronic® (BASF-Wyandotte, USA), are thermal setting polymers formed by a central hydrophobic part (polyoxypropylene) surrounded by a hydrophilic part (ethylene oxide). (4-(4,6-dimethoxy-1,3,5-triazin-2-yyl)-4methylmorpholinium choloride (DMTMM) or N-3-dimethylaminopropyl)-N′-ethylcarbodiimide hydrochloride and N-hydroxysuccinimide (EDC/NHS) may be useful to synthesize hyaluronan derivatives. See, D'Este, M. et al, Carbohydrate Polymers (2014) 108: 239-246). Cellulosic derivatives most commonly used in ophthalmology include: methylcellulose; hydroxyethylcellulose (HEC), hydroxypropylcellulose (HPC), hydroxypropylmethylcellulose (HPMC) and sodium carboxymethylcellulose (CMC Na). Photocrosslinked poly(ethylene glycol) diacrylate (PEGDA) hydrogels displaying collagen mimetic peptides (CMPs) that can be further conjugated to bioactive molecules via CMP-CMP triple helix association are described in Stahl, P J et al. Soft Matter (2012) 8: 10409-10418.

According to some embodiments, a first polymer and a second polymer comprise one or more different non-repeating units, such as, for example, an end group, or a non-repeating unit in the backbone of the polymer. According to some embodiments, the first polymer and the second polymer comprise one or more different end groups. For example, the first polymer can have a more polar end group than one or more end group(s) of the second polymer. According to some such embodiments, the first polymer will be more hydrophilic, relative to a second polymer (with the less polar end group) alone. According to some such embodiments, the first polymer comprises one or more carboxylic acid end groups, and the second polymer comprises one or more ester end groups.

According to some embodiments, the inlay material comprises a polymer matrix.

According to some embodiments, the inlay materials comprise an ultraviolet blocker.

The corneal inlay 10 can have properties similar to those of the cornea in nature, and may be made of a hydrogel or other clear biocompatible material. To increase the optical power of the inlay, the inlay may be made of a material with a higher index of refraction than the cornea, e.g., >1.376.

Materials that can be used to make the cornea inlay 10 include, but are not limited to, a self-assembling peptide hydrogel containing one or more non-protein amino acids (e.g., Thota, C K et al, Sci. Rep. 6: 31167; doi: 10.1038/srep31167 (2016), collagen mimetic peptide (“CMP”) conjugated with polyethylene glycol (“PEG”), lidofilcon A (a high water (>50% water nonionic hydrogel polymer), poly(2-hydroxyethyl methacrylate) (PolyHEMA), polysulfone, a silicone hydrogel polymer, water, and the like.

According to some embodiments, the composition of the corneal inlay 10 comprises water and CMP conjugated with PEG. According to some embodiments, the composition of the corneal inlay 10 comprises water, one or more hydrophilic polymers (e.g., PEG, MPC), and a mammalian collagen.

According to some embodiments, the water content can range from 80%-99%. According to some embodiments, the water content is at least 80%. According to some embodiments, the water content is at least 81%. According to some embodiments, the water content is at least 82%. According to some embodiments, the water content is at least 83%. According to some embodiments, the water content is at least 84%. According to some embodiments, the water content is at least 85%. According to some embodiments, the water content is at least 86%. According to some embodiments, the water content is at least 87%. According to some embodiments, the water content is at least 88%. According to some embodiments, the water content is at least 89%. According to some embodiments, the water content is at least 90%. According to some embodiments, the water content is at least 91%. According to some embodiments, the water content is at least 92%. According to some embodiments, the water content is at least 93%. According to some embodiments, the water content is at least 94%. According to some embodiments, the water content is at least 95%. According to some embodiments, the water content is at least 96%. According to some embodiments, the water content is at least 97%. According to some embodiments, the water content is at least 98%. According to some embodiments, the water content is at least 99%. According to some embodiments, the water content, by way of example, is at least 90%.

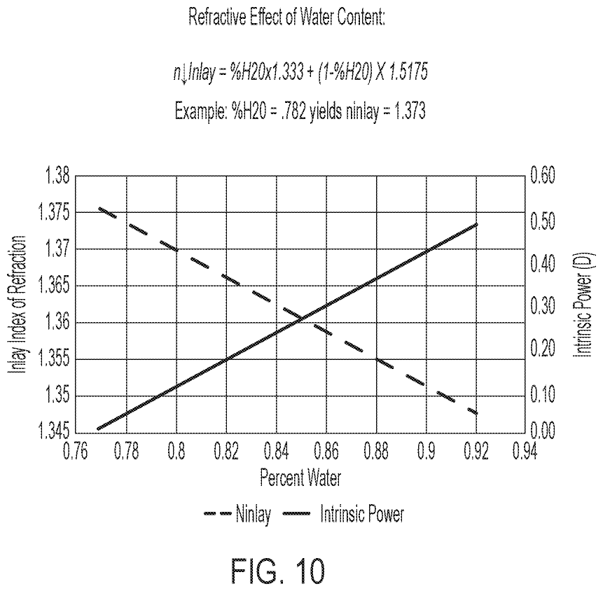

FIG. 10 is a graph showing the refractive effect of water content to an inlay index of refraction and to an intrinsic power. As seen, intrinsic power increases as water percent increases, while the inlay index of refraction decreases as water percentage increases.

### Fabrication.

According to some embodiments, a reusable mold comprises a first mold half comprising a first mold surface in contact with a polymerizable and/or crosslinkable silicone containing the inlay forming composition and a second mold half comprising a second mold surface in contact with the inlay-forming composition. The first mold half and the second mold half may be configured to receive each other such that a cavity is formed between the first mold surface and the second mold surface. The cavity may define the shape of an inlay to be molded.

According to some embodiments, polymers can be injected into molds and corneal inlays then polymerized by a method appropriate for the particular polymer employed, e.g., chemically, by successive cross-linking of precursors using cross-linking agents, thermally, or by photopolymerization. After polymerization, the inlay can be removed from the mold (demolded), washed and stored in buffer with a preservative until use.

According to some embodiments, the corneal inlay is cast as a flat, thin, round disc. According to some embodiments, the fabricated inlay is cast as a hemispherical dome. According to some embodiments, the fabricated inlay is cast as a spherical lens.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1. Evaluating Corneal Haze after Corneal Implants of Various Inlay Materials in New Zealand White Rabbits (Non-GLP)

Background. This study is designed to evaluate corneal haze after corneal implants of various inlay materials in rabbits. This information can only be obtained from living systems that were treated with the inlay materials. The rabbit is a standard species used in ocular studies based upon historical data and FDA requirements. New Zealand White (NZW) rabbits, which are recognized as a preferred and optimal model for assessing study endpoints, have proven to be useful in ophthalmic research. Their ocular anatomy and physiology are similar to humans, and their eyes have similar metabolic pathways.

A. Proposed Study Duration: 5-6 Months

B. Experimental Design
1. Test system
   Species: Oryctolagus cuiculus
   Strain: New Zealand White rabbits
   Sex: Male (all same sex)
   Weight: Approximately 3.5 to 4.5 kg at study start
   Number: 16
   Method of identification: ear tag and cage label per SOPs
   Minimal acclimation: 5 days
2. Specialized animal husbandry and/or restraint
   (a) fasting: none
   (b) restraint: animals will be manually restrained per SOPs to facilitate examinations.
   (c) Housing: Animals will be singly housed prior to and during the study in order to decrease the likelihood of ocular injuries from cage mates.

C. Test Articles

| Test | | | ID | | | Expiry or |
|---|---|---|---|---|---|---|
| Article | Abbreviation | Description | No. | Manufacturer | Storage | retest date |
| 1 | PEG | Polyethylene Glycol (PEG); | 1386B | Ferentis | Room | N/A |
| | | 82% Water Content; | | | temperature | |
| | | (Diameter: ~2.5 mm inlay, | | | | |
| | | thickness: ~40 microns) | | | | |
| 2 | MPC | 2-methacryloyloxyethyl | 1385A | Ferentis | Refrigerated | N/A |
| | | phosphorylcholine polymer; | | | at 2-8° C. | |
| | | 82% Water Content; | | | | |
| | | (Diameters - 2.5 mm disc, | | | | |
| | | thickness: ~40 microns) | | | | |
| 3 | PEG-(CMP- | Collagen Mimetic | 1444A | Ferrentis | Refrigerated | N/A |
| | RGD)-MPC | Peptide with RGD motif- | | | at 2-8° C. | |
| | | 2-methacryloyloxyethyl | | | | |
| | | phosphorylcholine; | | | | |
| | | 80% Water Content; | | | | |
| | | (Diameter: ~2.5 mm disc, | | | | |
| | | thickness: ~36 microns) | | | | |
| 4 | PC-MPC | Porcine Collagen/ | 1442B | Ferentis | Refrigerated | N/A |
| | | 2-methacryloyloxethyl | | | at 2-8° C. | |
| | | phosphorylcholine | | | | |
| | | (PC-MPC) polymer; | | | | |
| | | 80% Water Content; | | | | |
| | | (Diameter: ~2.5 mm disc, | | | | |
| | | thickness: ~40 microns) | | | | |
| 5 | FIB-PEG- | Fibronectin-Polyethylene- | 1441 | Ferrentis | Refrigerated | N/A |
| | CMP-MPC | glycol-Collagen Mimetic | | | at 2-8° C. | |
| | | Peptide-2-methacryloyloxethyl | | | | |
| | | phosphorylcholine; | | | | |
| | | 80% Water Content; | | | | |
| | | (Diameter: ~2.5 mm disc, | | | | |
| | | thickness: ~32 microns) | | | | |
| 6 | Biotrue | 22% Nesofilcon A; | TBD | Optics | Refrigerated | N/A |
| | | 78% Water Content; | | Medical | at 2-8° C. | |
| | | (Diameter: ~2.5 mm disc, | | | | |
| | | thickness: ~45 micron)′ | | | | |
| 7 | PC-MPC | PorcineCollagen/ | 1366B | Ferrentis | Refrigerated | N/A |
| Control | | 2-methacryloyloxethyl | | | at 2-8° C. | |
| Article 1 | | phosphorylcholine | | | | |
| | | (PC-MPC) polymer; | | | | |
| | | 90% Water Content; | | | | |
| | | (Diameter: ~2.5 mm disc, | | | | |
| | | thickness: ~40 microns) | | | | |
| 8 | Raindrop | Raindrop - Near Vision Inlay; | 003450 | Revision | Room | 2021 Jan. 16 |
| Control | Inlay | 78% Water Content; | | Optics | Temperature | |
| Article 2 | | (Diameter: ~2.0 mm inlay, | | | | |
| | | thickness: ~34 microns) | | | | |

### Details of Test Article Administration

Pre-Treatment Examinations

Prior to placement on study, each animal will undergo an ophthalmic examination (slit-lamp biomicroscopy and indirect ophthalmoscopy) to be performed by the Study Director or the Associate Director. Ocular findings will be scored according to a modified McDonald-Shadduck Scoring System (Appendix A). The acceptance criteria for placement on study will be scores of “0” for all variables.

Anesthesia

Animals will be anesthetized via an IM injection of a cocktail containing ketamine (up to approximately 50 mg/kg), glycopyrrolate (0.01 mg/kg, IM) and xylazine (up to approximately 10 mg/kg). Atipamezole hydrochloride (up to 1 mg/kg) may be used as a reversal agent. One to two drops of topical proparacaine hydrochloride anesthetic (0.5%) will be applied to the animals' eyes prior to the injection procedure. Additional topical ocular anesthesia dosing may be utilized during the procedure if needed.

Surgical Procedure for Nictitating Membrane Removal

Due to the ability of the nictitating membrane, or third eyelid, to push out the test article, each rabbit will have the nictitating membrane removed from both eyes prior to film placement. Since humans do not have nictitating membranes, removal of these membranes provides a model that more closely mimics human eyes. Nictitating membranes will be removed at least 10 days prior to test article administration.

Animals will be anesthetized as described above. Both eyes of each rabbit will be cleaned with betadine and then rinsed with balanced salt solution (BSS). One to two drops of topical proparacaine hydrochloride anesthetic (0.5%) will be applied to the nictitating membrane of each of the animal's eyes prior to the surgical procedure.

The nictitating membrane will be grasped with a pair of forceps and gently clamped at its base with a pair of hemostats. After clamping for approximately 1 to 2 minutes, the clamp will be removed and the nictitating membrane excised along the clamp line with scissors according to SOP ASI-112. The area may be blotted and medicated with topical gentamicin (0.3%) and neodecadron (1 to 2 drops). The contralateral eye will have its nictitating membrane removed with the same procedure. Triple antibiotic ointment will be applied topically once immediately following nictitating membrane removal. Post-operative recovery for the rabbits will be as described in SOPs ASI-079, ASI-057, and ASI-102 (if catheter placement is necessary). One injection of buprenorphine (0.02 to 0.05 mg/kg, IM/SC) may be given once following removal of the nictitating membranes if deemed necessary by the Attending Veterinarian. Any analgesic treatments will be administered to all study animals equally.

The day after surgery, animals will be examined to ensure there were no post surgical complications. Animals will receive triple antibiotic ointment for up to 3 days post-operatively. Additional buprenorphine may be administered as deemed necessary by the Attending Veterinarian (this could be more than once). If post-surgical complications occur, the Study Director and/or veterinary staff will be consulted as to the appropriate course of action to maintain the animal's health and well-being. The eyes will be allowed to heal for at least 10 days prior to the administration of the test article as described below.

Surgical Procedure for Test Article Administration

Test articles will be implanted in the corneas of both eyes of all study animals on Day 0 according to the study design in Table 1. Implantation procedures will be performed by the designated surgeon. Laser, microkeratome and surgical supplies will be provided by the Sponsor.

Animals will be anesthetized as outlined above. The eyes will be cleaned with betadine and then rinsed with BSS.

A flap or pseudo-pocket will be cut into each cornea using a laser or a microkeratome. The surgery type will be noted in the study data.

The appropriate inlay for each eye will be inserted into the flap or pseudo-pocket. Inlays will be stained with 25% fluorescein (provided by the Sponsor) to facilitate visualization during the implantation procedure.

After the surgical procedures, animals will be recovered from anesthesia per ASC SOPs.

Analgesics (e.g. buprenorphine [0.01 to 0.05 mg/kg, IM/SC]), antibiotics (e.g., triple antibiotic ointment or 0.3% tobramycin drops), and prednisolone as an anti inflammatory treatment will be administered on Days 1-3 after the surgical procedures as deemed necessary by the Study Director and/or the Attending Veterinarian. Analgesic, anti-inflammatory, and/or antibiotic regimens may be extended or otherwise modified as necessary based on the discretion of the Study Director and/or the Attending Veterinarian. Any such treatments will be recorded in the raw data.

### Safety Precautions

Standard laboratory safety procedures will be employed for handling the test articles. Specifically, gloves and lab coat along with appropriate vivarium attire will be worn while preparing and administering dose

TABLE 1
Study Design

| | | | Surgery | Slit-lamp | Corneal | Euthanasia and |
|---|---|---|---|---|---|---|
| Animal # | Eye | Materials | Technique * | Examinations | OCT | Tissue Collection |
| 1 | OS | RAINDROP | Flap | Baseline^{¥} and | Baseline | Day 180(±4)^{#α}: |
| | OD | PC-MPC 80% H₂O | | Days 7, | and Days | Whole Globes |
| 2 | OS | RAINDROP | Pseudo | 30(±2), | 14(±1) and | |
| | OD | PC-MPC 80% H₂O | Pocket | 60(±2), | 90(±4). | |
| 3 | OS | PC-MPC 90% H₂O | Flap | 0(±4), | Optional: | |
| | OD | PC-MPC 80% H₂O | | 120(±4), | additional | |
| 4 | OS | PC-MPC 90% H₂O | Pseudo | 150(±4), and | OCT | |
| | OD | PC-MPC 80% H₂O | Pocket | 180(±4) | images as | |
| 5 | OS | PEG 80% H₂O | Flap | | requested | |
| | OD | PEG 80% H₂O | | | by the | |
| 6 | OS | PEG 80% H₂O | Pseudo | | sponsor. | |
| | OD | PEG 80% H2O | Pocket | | OCT will | |
| 7 | OS | BIOTRUE | Flap | | be | |
| | OD | PC-MPC 90% H₂O | | | performed | |
| 8 | OS | BIOTRUE | Pseudo | | prior to | |
| | OD | PC-MPC 90% H₂O | Pocket | | termination | |
| 9 | OS | MPC 82% H₂O | Flap | | | |
| | OD | | | | | |
| 10 | OS | MPC 82% H2O | Pseudo | | | |
| | OD | | Pocket | | | |
| 11 | OS | PEG-CMP-RGD- | Flap | | | |
| | OD | MPC 80% H₂O | | | | |
| 12 | OS | PEG-CMP-RGD- | Pseudo | | | |
| | OD | MPC 80% H₂O | Pocket | | | |
| 13 | OS | FIB-PEG-CMP-MPC | Flap | | | |
| | OD | 80% H₂O | | | | |
| 14 | OS | FIB-PEG-CMP-MPC | Pseudo | | | |
| | OD | 80% H₂O | Pocket | | | |
| 15 | OS | LASER—SHAM | Flap | | | |
| | OD | LASER—SHAM | Pseudo | | | |
| | | | Pocket | | | |
| 16 | OS | LASER—SHAM | Flap | | | |
| | OD | LASER—SHAM | Pseudo | | | |
| | | | Pocket | | | |

OD: right eye; OS: left eye
* Surgery Type (laser flap, laser pseudo pocket, or a microkeratome flap) may change at the Sponsor’s discretion.
^{¥}Indirect ophthalmoscopy only for baseline.
^{#}Optional extensions with monthly examinations past Day 180(±4) for some or all animals may be added at the Sponsor’s discretion.
^{α}Animals may be euthanized earlier and tissues collected in case of corneal damage developing.

In-Life Observations and Measurements Summary of Key Study Parameters is Presented in Appendix A.

Body Weights

Animals will be weighed prior to test article administration and termination.

General Health Observations

Animals will be observed within their cages once daily throughout the study period. Each animal will be observed for changes in general appearance and behavior. Any abnormal observations will be reported to the Study Director.

General health observations will be performed and recorded daily starting on Day 0 and continuing throughout the duration of the study. Health observations will include assessment of ocular abnormalities such as discharge, swelling, or hyperemia.

Slit-Lamp Examinations

Slit-lamp examinations will be performed at baseline prior to test article administration, and on Days 7, 30(±2), 60(±2), and 90(±4), 120(±4), 150(±4), and 180(±4) after test article administration.

Additional monthly examinations past Day 180(±4) may be added as an optional extension at the discretion of the Sponsor.

Slit-lamp examinations will be performed by the Study Director or the Associate Director and will assess only the ocular observation variables of the modified McDonald-Shadduck Scoring System (Appendix A) related to corneal haze/opacity. Severity (“Cornea”) and area (“Surface Area of Cornea Involvement”) of corneal haze/opacity will be assessed.

In addition, examinations will include scoring of corneal haze following the study-specific scoring system presented in Appendix B.

OCT Imaging

Optical coherence tomography (OCT) images will be taken at baseline and on Days 14(±1) and 90(±4) or prior to termination. More OCT time points may be added at the Sponsor's discretion.

OCT will be used to analyze the cornea cross-section, device placement, and geometry. Images will be taken to capture any ocular anomalies noted at the time of imaging. OCT examinations will be performed by the Study Director. All raw images taken will be provided to the Sponsor.

Animals may be anesthetized for OCT imaging as described above.

### Calculations and Statistical Analysis

Data will be presented in tabular format and no calculations or statistical analysis will be performed on the data collected during the in-life portion of the study.

### Terminal Procedures

Early Death/Unscheduled Sacrifice

If an animal dies on study, the time of death will be estimated as closely as possible and recorded. The animal may be necropsied; if so, the necropsy will be performed as soon as possible. If the necropsy cannot be performed immediately, the animal will be refrigerated (not frozen) to minimize tissue autolysis. Animals that are prematurely terminated may be necropsied after discussion with the Sponsor, and major organ findings noted.

If an animal is moribund as defined by SOP ASI-023 Care and Use of Animals, it will be euthanized as described below, which is in accordance to ASC's policies on humane care of animals. If an animal possesses any of the following signs it will be considered as indicative of moribund condition: impaired ambulation which prevents the animal from reaching food or water, excessive weight loss and emaciation (>20%), lack of physical or mental alertness, difficult labored breathing, or inability to remain upright. Animals with other less severe clinical signs will be treated (antibiotics or analgesics, fluids, etc.) or euthanized after discussion with Attending Veterinarian and Study Director. Any alternate endpoints (e.g. death, allowing ill animals to remain untreated and alive (i.e. moribund endpoints), etc.) must be justified in study documentation.

If possible, blood or other specimens may be collected and analyzed as appropriate (e.g., for clinical pathology parameters) to help reveal the cause of malaise/morbidity. All unscheduled-sacrifice animals may be necropsied. If so, necropsy will be performed immediately, or, if this cannot be performed, the animal will be refrigerated to minimize autolysis and necropsied no later than 12 hours after death. All tissues listed in this protocol will be preserved.

Specific ocular endpoints necessitating treatment and/or euthanasia would be:

Ocular infection

Ocular hemorrhage, hyperemia

Visual impairment that is manifesting in behavior abnormalities, pain, and/or distress to the animal

Loss of globe integrity

Corneal damage

In the event that an animal dies or is euthanized during the study, terminal procedures will be conducted as per SOPs.

Euthanasia

After completion of the final examination on Day 90 (±4), animals will be euthanized by an intravenous injection of a commercial barbiturate based euthanasia solution (approximately 150 mg/kg, to effect). If there is an indication of corneal damage, animals may be euthanized earlier and tissues will be collected. At the Sponsor's discretion, Day 90 (±4) euthanasia may be extended with weekly examinations. The euthanasia procedure will be performed in compliance with the 2013 American Veterinary Medical Association (AVMA) Guidelines on Euthanasia.

Tissue Collection: Tissues will be collected according to the method described below.

Method 1

Immediately following euthanasia, the anterior chamber of the eye will be perfused with 2% paraformaldehyde (PFA) in phosphate-buffered saline (PBS), pH 7.4, for 4 minutes to fix the cornea. Perfusion will be performed using handheld syringes using a push-pull technique (two needles, one for pushing in PFA, one for pulling out aqueous humor). A slow push/pull will be used to maintain the internal pressure of the eye and avoid damage to the cornea. After perfusion, the eye will be harvested. The cornea plus 1-2 mm limbal tissue will be excised using scalpel puncture and curved corneal scissors. The remaining eye will be discarded.

The excised cornea will be placed into a chilled container with 2% PFA. The container will be sealed to prevent leakage or evaporation and immediately placed on wet ice until being stored refrigerated at 2-8° C.

Samples collected via this method will be shipped on cold packs via overnight shipment within 2 days of collection (to ensure receipt of samples within 3 days of collection) to the Sponsor's designated laboratory.

Method 2

Immediately following euthanasia, the eye will be harvested. The whole globe will be placed in the Davidson's solution immediately after trimming excessive tissues. A gauze pad should be used to keep the eye submerged if necessary for consistent fixation. Keep the globe in the Davidson's solution for 48 hours. The eye then is taken out of the solution and placed in 70% ethanol.

Samples collected via this method will be shipped in 70% ethanol solution to the Sponsor's designated laboratory.

A total of 32 corneas will be collected.

### APPENDIX A. MODIFIED MEDONALD-SHADDUEK SCORING SYSTEM

(T. McDonald and J. A. Shadduck, “Eye irritation,” in Advances in Modern Toxicology: Dermatoxicology, F. Marzulli and H. I. Maibach, Eds., pp. 579-582, Hemisphere Publishing Corporation, Washington, D.C., USA, 1977)

Examination:

Use the slit lamp to observe the following:
Pupillary Response
Conjunctival Discharge
Conjunctival Congestion
Conjunctival Swelling
Cornea
Surface Area of Cornea Involvement
Pannus
Aqueous Flare
Aqueous Cell
Iris Involvement
Lens

Use the Indirect Ophthalmoscope for the following:
Vitreous Flare
Vitreous Cell
Vitreal Hemorrhage
Retinal Detachment
Retinal Hemorrhage
Choroidal/Retinal Inflammation

Prepare animal for observation by using one of three solutions to dilate the pupil. Usually two drops of ophthalmic preparations of atropine, tropicamide, or phenylephrine is sufficient. The choice of dilator will generally be outlined in the study protocol. Wait until pupil of animal appears to be dilated. It may take up to 60 minutes to achieve pupil dilation.

Pupillary Response: Check for any blockage or a sluggish response in the pupillary region. Scoring will be taken as follows:
0=Normal pupil response.
1=Sluggish or incomplete pupil response.
2=No pupil response.
3=No pupil response due to pharmacological blockage.

Conjunctival Discharge: Discharge is defined as a whitish gray precipitate from the eye. Scoring will be taken as follows:
0=Normal. No discharge.
1=Discharge above normal and present on the inner portion of the eye but not on the lids or hairs of the eyelids.
2=Discharge is abundant, easily observed and has collected on the lids and hairs of the eyelids.
3=Discharge has been flowing over the eyelids so as to wet the hairs substantially on the skin around the eye.

Conjunctival Congestion: Congestion causes the blood vessels of the eye to become enlarged. Scoring will be taken as follows:
0=Normal. May appear blanched to reddish pink without perilimbal injection (except at the 12:00 and 6:00 positions) with vessels of the palpebral and bulbar conjunctiva easily observed.
1=A flushed, reddish color predominantly confined to the palpebral conjunctiva with some perilimbal injection but primarily confined to the lower and upper parts of the eye from the 4:00 to 7:00 and 11:00 to 1:00 positions.
2=Bright red color of the palpebral conjunctiva with accompanying perilimbal injection covering at least 75% of the circumference of the perilimbal region.
3=Dark, beefy red color with congestion of both the bulbar and palpebral conjunctiva along with pronounced perilimbal injection and the presence of petechia on the conjunctiva. The petechia generally predominates along the nictitating membrane and upper palpebral conjunctiva.

Conjunctival Swelling (meaning swelling of the conjunctiva). Scoring will be taken as follows:
0=Normal or no swelling of the conjunctival tissue
1=Swelling above normal without eversion of the eyelids (easily discerned by noting upper and lower eyelids are positioned as in the normal eye); swelling generally starts in the lower cul-de-sac near the inner canthus.
2=Swelling with misalignment of the normal approximation of the lower and upper eyelids; primarily confined to the upper eyelid so that in the initial stages, the misapproximation of the eyelids begins by partial eversion of the upper eyelid. In this stage the swelling is confined generally to the upper eyelid with some swelling in the lower cul-de-sac.
3=Swelling definite with partial eversion of the upper and lower eyelids essentially equivalent. This can be easily observed by looking at the animal head-on and noting the position of the eyelids; if the eye margins do not meet, eversion has occurred.
4=Eversion of the upper eyelid is pronounced with less pronounced eversion of the lower eyelid. It is difficult to retract the lids and observe the perilimbal region.

Cornea: Check the Cornea for any abnormalities. Scoring will be taken as follows:
0=Normal Cornea
1=Some loss of transparency. Only the epithelium and/or the anterior half of the stroma are involved. The underlying structures are clearly visible although some cloudiness may be readily apparent.
2=Involvement of the entire thickness of the stroma. With diffuse illumination, the underlying structures are just barely visible (can still observe flare, iris, pupil response, and lens).
3=Involvement of the entire thickness of the stroma. With diffuse illumination, the underlying structures cannot be seen.

0=Normal Surface Area of Cornea Involvement: Check the eye for cloudiness in the stromal region. Scoring will be taken as follows:
1=1-25% area of stromal cloudiness.
2=26-50% area of stromal cloudiness.
3=51-75% area of stromal cloudiness.
4=76%-100% area of stromal cloudiness.

Pannus: Check for vascularization of Cornea. Scoring will be taken as follows:
0=No pannus (vascularization of the cornea)
1=Vascularization present but vessels have not invaded the entire cornea circumference.
2=Vessels have invaded 2 mm or more around entire corneal surface.

Aqueous Flare: Breakdown of the blood-aqueous barrier. Field size is a 1 mm×1 mm slit beam. Scoring will be taken as follows (based on Jabs D A et al., 2005):
0=None
1=Faint
2=Moderate (iris and lens details clear)
3=Marked (iris and lens details hazy)
4=Intense (fibrin or plastic aqueous)

Aqueous Cell: Cellular observation in the aqueous humor. Field size is a 1 mm×1 mm slit beam. Scoring will be taken as follows (based on Jabs D A et al., 2005):
0=None
0.5=Trace (1-5)
1=6-15
2=16-25
3=26-50
4=>50

Iris Involvement: Check the iris for hyperemia of the blood vessels. Scoring will be taken as follows:
0=Normal iris without any hyperemia of the blood vessels.
1=Minimal injection of the secondary vessels but not tertiary vessels. Generally uniform but may be of greater intensity at the 12:00 to 1:00 or 6:00 position. If confined to this area, the tertiary vessels must be substantially hyperemic.
2=Minimal injection of tertiary vessels and minimal to moderate injection of the secondary vessels.
3=Moderate injection of the secondary and tertiary vessels with slight swelling of the iris stroma (the iris surface appears slightly rugose, usually most predominant near the 3:00 and 9:00 positions).
4=Marked injection of the secondary and tertiary vessels with marked swelling of the iris stroma. The iris appears rugose; may be accompanied by hemorrhage (hyphema) in the anterior chamber.

Lens: Observe the lens for any cataracts. Scoring will be taken as follows:
0=Lens clear.
1=Anterior (cortical/capsular).
2=Nuclear.
3=Posterior (cortical/optical).
4=Equatorial.

Vitreous Flare: Opacity or fogginess of the vitreous humor. Scoring will be taken as follows (based on Opremcak E M, 2012):
0=None (nerve fiber layer [NFL] clearly visible)
1=Faint (optic nerve and vessels clear, NFL hazy)
2=Moderate (optic nerve and vessels hazy)
3=Marked (optic nerve only visible)
4=Intense (no optic nerve visible)

Vitreous Cell: Cellular observation in the vitreous humor. Scoring will be taken as follows (based on Opremcak E M, 2012):
0=Trace (0-10)
1=11-20
2=21-30
3=31-100
4=>100

Vitreal Hemorrhage: Observe the vitreous for any hemorrhage. Scoring will be taken as follows:
0=None
1=1-25%
2=26-50%
3=51-75%
4=76-100%

Retinal Detachment: During a retinal detachment, bleeding from small retinal blood vessels may cloud the interior of the eye, which is normally filled with vitreous fluid. Scoring will be taken as follows:
0=None
1=Rhegmatogenous (retinal detachment occurs when subretinal fluid accumulates in the potential space between the neurosensory retina and the underlying retinal pigment epithelium).
2=Exudative (occurs due to inflammation, injury, or vascular abnormalities that results in fluid accumulating underneath the retina without the presence of a hole, tear, or break).
3=Tractional (occurs when fibrous or fibrovascular tissue, caused by an injury, inflammation, or neovascularization that pulls the sensory retina from the retinal pigment epithelium).

Retinal Hemorrhage: Abnormal bleeding of the blood vessels in the retina. Scoring will be taken as follows:
0=None
1=1-25%
2=26-50%
3=51-75%
4=76-100%

Choroidal/Retinal Inflammation: Inflammation of the retina and/or choroid. Scoring will be taken as follows:
0=None
1=Mild
2=Moderate
3=Severe

### REFERENCES

Jabs D A, Nussenblatt R B, Rosenbaum J T, Standardization of Uveitis Nomenclature (SUN) Working Group (2005). Standardization of uveitis nomenclature for reporting clinical data. Results of the First International Workshop. American Journal of Ophthalmology 140(3): 509-516.
Opremcak E M (2012). Uveitis: A Clinical Manual for Ocular Inflammation. New York: Springer Science+Business Media.

### APPENDIX B: CORNEAL HAZE SCORING

Haze grading is based on a scale used to grade post-PRK Haze, Arch. Ophthalmology (1992) (110): 1286-1291):

Clear (Grade 0): sporadic, peripheral faint haze, (CLEAR CENTER), not visible with diffuse slit lamp beam, minimally visible by oblique or slit beam. Vision is not affected.

Trace Haze (Grade 1): Trace haze covering mid-peripheral and center of inlay. Visible with difficulty using diffuse illumination, visible by broad tangential illumination. May present with myopic shift, reduced hear point, visual symptoms (glare and halo).

Mild (Grade 2)

Moderate (Grade 3)

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

1) A method of treating presbyopia comprising placing in a cornea of a mammalian subject a corneal inlay device of high water content the corneal inlay device comprising a thickness, a diameter, a flat or flat-like base and a dome or droplet shaped top, the dome or droplet shaped top forming a contact angle with the base, wherein the corneal inlay device, when placed in the cornea is effective: to alter a shape of the anterior surface of a cornea, and to increase an eye's ability to increase its power to focus on nearby objects, with a reduced risk of development of corneal haze compared to a control.

2) The method of claim 1, wherein the placing of the corneal inlay device is by cutting a flap in the cornea and positioning the inlay beneath the flap.

3) The method of claim 1, wherein the placing of the corneal inlay device is by positioning the inlay device within a pocket formed in the cornea.

4) The method of claim 1, wherein the placing of the corneal inlay device is in the cornea at a depth of about 100 microns to about 200 microns, inclusive.

5) The method of claim 1, wherein the placing of the corneal inlay device is in the cornea at a depth of about 130 microns to about 160 microns, inclusive.

6) The method of claim 1, wherein the contact angle is between 1° and 180°.

7) The method of claim 1, wherein the thickness of the corneal inlay ranges from at least 25 microns, at least 26 microns, at least 27 microns, at least 28 microns, at least 29 microns, at least 30 microns, at least 31 microns, at least 32 microns, at least 33 microns, at least 34 microns, at least 35 microns, at least 36 microns, at least 37 microns, at least 38 microns, at least 39 microns, at least 40 microns, at least 41 microns, at least 42 microns, at least 43 microns, at least 44 microns, at least 45 microns, at least 46 microns, at least 47 microns, at least 48 microns, at least 49 microns, at least 50 microns, at least 51 microns, at least 52 microns, at least 53 microns, at least 54 microns, at least 55 microns, at least 56 microns, at least 57 microns, at least 58 microns, at least 59 microns, to 60 microns.

8) The method of claim 5, wherein the thickness of the corneal inlay ranges from at least 32 microns, at least 33 microns, at least 34 microns, at least 35 microns, at least 36 microns, at least 37 microns, at least 38 microns, at least 39 microns, at least 40 microns, at least 41 microns, at least 42 microns, at least 43 microns, at least 44 microns, at least 45 microns, at least 46 microns, at least 47 microns, at least 48 microns, at least 49 microns, to 50 microns.]

9) The method of claim 1, wherein diameter of the corneal inlay device is at least 1 mm, at least 1.1 mm, at least 1.2 mm, at least 1.3 mm, at least 1.4 mm, at least 1.5 mm, at least 1.6 mm, at least 1.7 mm, at least 1.8 mm, at least 1.9 mm, at least 2.0 mm, at least 2.1 mm, at least 2.2 mm, at least 2.3 mm, at least 2.4 mm, at least 2.5 mm, at least 2.6 mm, at least 2.7 mm, at least 2.8 mm, at least 2.9 mm, or at least 3.0 mm.

10) The method of claim 1, wherein the corneal inlay device comprises water, a hydrophilic polymer, and a protein.

11) The method of claim 10, wherein the protein is an isolated protein, a recombinant protein, a synthetic protein, or a peptidomimetic.

12) The method of claim 10, wherein the hydrophilic polymer comprises polyethylene glycol (“PEG”), poly(2-methacryloyloxyethyl phosphorylcholine) (MPC), or both.

13) The method of claim 1, wherein water content of the corneal inlay is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%.

14) The method of claim 1, wherein the corneal inlay device is optically transparent, biocompatible, permeable and refractive.

15.-28. (canceled)
